(19) United States
(12) Patent Application Publication
(10) Pub. No.: US 2024/0024156 A1
(43) Pub. Date: Jan. 25, 2024

(54) HEAT MANAGEMENT OF PROSTHESES

(71) Applicant: COCHLEAR LIMITED, Macquarie University, NSW (AU)

(72) Inventors: Guilhem Yvan VAVELIN, Macquarie University (AU); Werner MESKENS, Macquarie University (AU)

(21) Appl. No.: 18/029,914

(22) PCT Filed: Oct. 1, 2021
(86) PCT No.: PCT/IB2021/059048

§ 371 Date: Apr. 2, 2023

Related U.S. Application Data
(60) Provisional application No. 63/086,356, filed on Oct. 1, 2020.

(51) Int. Cl. A61F 7/00; A61N 1/36; A61B 5/00

(52) U.S. Cl. CPC A61F 7/007; A61N 1/36038; A61B 5/00; A61B 2560/0219; A61F 2007/0005

(57) ABSTRACT

A device, including an inductive power transmission apparatus, wherein the device includes a dedicated heat transfer arrangement configured to transfer away from the device heat that is generated when transferring power using the device, and the device is configured to transmit inductance power transcutaneous into a person. The device can be a hearing prosthesis component.

### CROSS-REFERENCE TO RELATED APPLICATIONS

This application claims priority to U.S. Provisional Application No. 63/086,356, entitled HEAT MANAGEMENT OF PROSTHESES, filed on Oct. 1, 2020, naming Guilhem Yvan VAVELIN of Mougins, France as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In accordance with an exemplary embodiment, there is a device, comprising an inductive power transmission apparatus, wherein the device includes a dedicated heat transfer arrangement configured to transfer away from the device heat that is generated when transferring power using the device, and the device is configured to transmit inductance power transcutaneous into a person.

In accordance with another exemplary embodiment, there is a method, comprising placing a transcutaneous power transfer apparatus at a location on a surface of the skin proximate an implanted medical device, transferring power from the apparatus to the implanted medical device and at least one of transferring heat away from the location while transferring power from the apparatus to the medical device or cooling the transcutaneous power transfer apparatus prior to transferring power from the apparatus to the medical device.

In another exemplary embodiment, there is a method, comprising obtaining a device configured to transcutaneously charge and/or power an implanted prosthesis implanted in a recipient, which device has a rechargeable power storage component from which power is extracted to charge and/or power the implanted prosthesis, the power storage device having a state of charge less than fully charged, and recharging the power storage component to elevate the state of charge of the power storage component, wherein the device is affirmatively cooled during the action of recharging so that an outer surface of the device that interfaces with skin of a person during charging and/or powering of the implanted prosthesis has a temperature that is lower than that which would otherwise be the case in the absence of the affirmative cooling.

In another exemplary embodiment, there is a device, comprising an inductive power transmission sub-system configured to transfer power to an implanted medical device, a skin interface surface and a cooling sub-system configured to cool the skin interface surface.

In another exemplary embodiment, there is a device, comprising a battery charging apparatus and a cooling device, wherein the device is a dedicated prosthesis component charging device configured to recharge a power storage portion of the prosthesis component while cooling an assembly of which the power storage portion is apart.

In another exemplary embodiment, there is a headpiece of a hearing prosthesis, comprising a DC battery, an inductive power driver including transistors, the inductive power driver being configured to use the transistors to convert the direct current of the battery to alternating current, a magnet and an inductive coil extending about the magnet, wherein the inductive coil is in electrical communication with the inductive power driver so that the inductive coil receives the alternating current and generates an inductance field to power an implantable hearing prosthesis, wherein the inductive coil made from a metal heat pipe that is configured to fluidically transfer heat away from the coil, which heat that is generated when transferring inductive power using the coil.

### BRIEF DESCRIPTION OF THE DRAWINGS

Embodiments of the present invention are described below with reference to the attached drawings, in which:

FIG. 1 is a perspective view of an exemplary hearing prosthesis in which at least some of the teachings detailed herein are applicable;

FIG. 1A is a view of an exemplary sight prosthesis in which at least some of the teachings herein are applicable;

FIG. 2 schematically illustrates another exemplary hearing prosthesis in which at least some of the teachings detailed herein are applicable;

FIG. 3 schematically illustrates another exemplary hearing prosthesis in which at least some of the teachings detailed herein are applicable;

FIG. 4 schematically illustrates another exemplary hearing prosthesis in which at least some of the teachings detailed herein are applicable;

FIG. 5 schematically illustrates another exemplary hearing prosthesis in which at least some of the teachings detailed herein are applicable;

FIG. 6 schematically illustrates another exemplary hearing prosthesis in which at least some of the teachings detailed herein are applicable;

FIGS. 7-9 depict functional diagrams associated with some embodiments;

FIG. 10 is a cross-section of an apparatus used in some embodiments;

FIGS. 11-13 and 16 schematically illustrates additional exemplary hearing prostheses in which at least some of the teachings detailed herein are applicable;

FIG. 14 presents an exemplary flowchart for an exemplary method;

FIG. 15 presents a diagram of a head of a human;

FIGS. 17, 18 and 19 presents additional exemplary flowcharts for exemplary methods; and

FIGS. 20 and 21 depict exemplary charging devices for prosthesis devices.

### DETAILED DESCRIPTION

Merely for ease of description, the techniques presented herein are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis). The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. It is also noted that in an exemplary embodiment, the teachings herein can be used with a retinal implant device. Thus, any disclosure herein corresponds to a disclosure of expanding functionality to include the functionality of a retinal implant, and, for example, any disclosure of a cochlear implant processor corresponds to a light processor. In further embodiments, the techniques presented herein may be used with air purifiers or air sensors (e.g., automatically adjust depending on environment), hospital beds, identification (ID) badges/bands, or other hospital equipment or instruments, where such rely upon behind the ear devices.

By way of example, any of the technologies detailed herein which are associated with components that are implanted in a recipient can be combined with information delivery technologies disclosed herein, such as for example, devices that evoke a hearing percept and/or devices that evoke a vision percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface as will be described herein that enables information to be conveyed to the recipient, which information is associated with the implant.

Moreover, embodiments need not necessarily provide input or status information to the recipient. Instead, the various sensors detailed herein can be utilized in combination with the nonsensory implants detailed herein so as to enable control or performance adjustments of the implanted component. For example, the embodiments that utilize sensors and the associated logic circuitry that would be combined with a sleep apnea device, for example, can be utilized to enable the recipient to input commands to control the implant. Such can potentially also be done with respect to a bionic arm or bionic leg, etc. In this regard, embodiments can enable a user interface that can enable a recipient to provide input into the prosthesis to control the prosthesis without utilizing any artificial external component. For example, embodiments can enable the input utilizing only the recipient's voice and/or only the recipient's hand/fingers. Thus, embodiments can enable control of such prostheses utilizing only a recipient's hand and/or only a recipient's voice. Accordingly, at least some exemplary embodiments can combine hearing prosthesis technology with the innovations detailed herein with other implant technologies to enable control without the need of other artificial devices.

Thus, the teachings detailed herein are implemented in sensory prostheses, such as hearing devices, including hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system, and/or method that is used to or results in ultimate stimulation of the auditory nerve corresponds to a disclosure of an analogous stimulation of the optic nerve utilizing analogous components/methods/systems. All of this can be separately or in combination.

Embodiments detailed herein focus on the utilization of a hearing prosthesis to provide status and information a recipient. It is to be understood that in some embodiments, a retinal prosthesis can be utilized to provide visual input to the recipient. By way of example only and not by way of limitation, in an exemplary embodiment, the retinal prosthesis can be configured to results in a vision of an artificial image, which can correspond to words or the like, which can correspond to a status of the prostheses. Accordingly, any disclosure herein associated with providing sound-based or hearing percept base information the recipient also corresponds to a disclosure of providing vision based information to the recipient and vice versa.

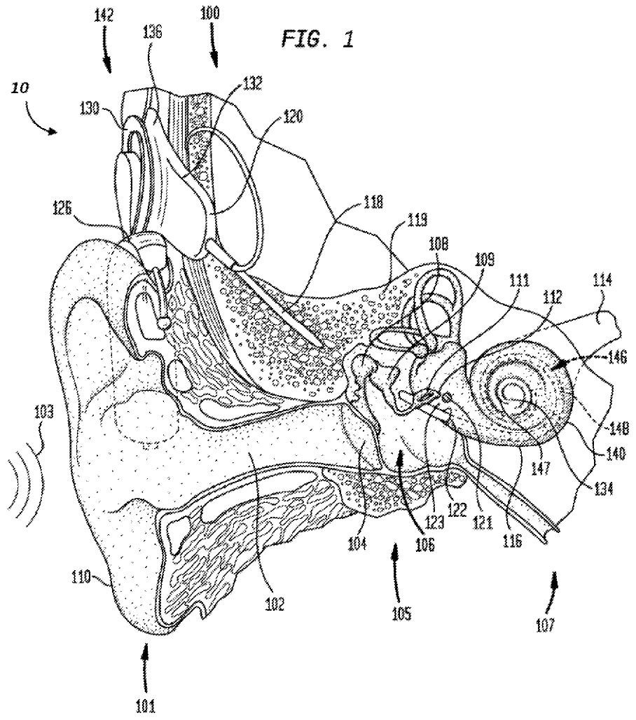

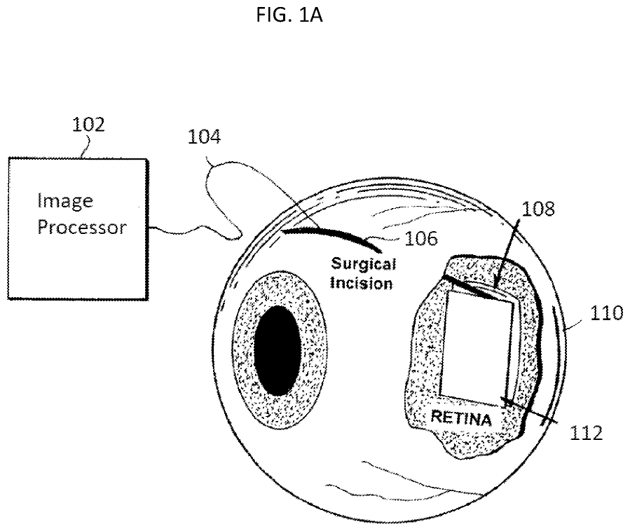

FIG. 1 is a perspective view of a totally implantable cochlear implant, referred to as cochlear implant 100, implanted in a recipient, to which some embodiments detailed herein and/or variations thereof are applicable. The totally implantable cochlear implant 100 is part of a system 10 that can include external components, in some embodiments, as will be detailed below. It is noted that the teachings detailed herein are applicable, in at least some embodiments, to any type of hearing prosthesis having an implantable microphone. The teachings detailed herein are also applicable, in at least some embodiments, to any type of hearing prosthesis not having an implantable microphone, and thus are applicable to non-totally implantable hearing prostheses.

It is noted that in alternate embodiments, the teachings detailed herein and/or variations thereof can be applicable to other types of hearing prostheses, such as, for example, bone conduction devices (e.g., active transcutaneous bone conduction devices), Direct Acoustic Cochlear Implant (DACI), etc. Embodiments can include any type of hearing prosthesis that can utilize the teachings detailed herein and/or variations thereof. It is further noted that in some embodiments, the teachings detailed herein and/or variations thereof can be utilized by other types of prostheses beyond hearing prostheses.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142. In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement. Some additional details associated with the implantable microphone assembly 137 will be detailed below.

Main implantable component 120 further includes a stimulator unit (not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments, electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

As noted above, cochlear implant 100 comprises a totally implantable prosthesis that is capable of operating, at least for a period of time, without the need for external device 142. Therefore, cochlear implant 100 can further comprise a rechargeable power source (not shown) that stores power received from external device 142. The power source can comprise, for example, a rechargeable battery. During operation of cochlear implant 100, the power stored by the power source is distributed to the various other implanted components as needed. The power source may be located in main implantable component 120, or disposed in a separate implanted location.

It is noted that the teachings detailed herein and/or variations thereof can be utilized with a non-totally implantable prosthesis. That is, in an alternate embodiment of the cochlear implant 100, the cochlear implant 100 is a traditional hearing prosthesis.

In some exemplary embodiments, a signal sent to the stimulator of the cochlear implant can be derived from an external microphone, in which case the system is called a semi-implantable device, or from an implanted microphone, which then refers to a fully implantable device. DACIs and other types of implants can also use an implanted microphone, and thus are also fully implantable devices. Fully implantable devices can have utility by presenting improved cosmesis, can have an improved immunity to certain noises (e.g., wind noise), can present few opportunities for loss or damage, and can at least sometimes be more resistant to clogging by debris or water, etc. DACIs can have utilitarian value by keeping the ear canal open, which can reduce the possibility of infection of the ear canal, which otherwise is humid, often impacted with cerumen (earwax), and irritated by the required tight fit of a non-implanted hearing aid.

FIG. 1A presents an exemplary embodiment of a neural prosthesis in general, and a retinal prosthesis and an environment of use thereof, in particular. In some embodiments of a retinal prosthesis, a retinal prosthesis sensor-stimulator 108 is positioned proximate the retina 110. In an exemplary embodiment, photons entering the eye are absorbed by a microelectronic array of the sensor-stimulator 108 that is hybridized to a glass piece 112 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 102 is in signal communication with the sensor-stimulator 108 via cable 104 which extends through surgical incision 106 through the eye wall (although in other embodiments, the image processor 102 is in wireless communication with the sensor-stimulator 108). In an exemplary embodiment, the image processor 102 is analogous to the sound processor/signal processors of the auditory prostheses detailed herein, and in this regard, any disclosure of the latter herein corresponds to a disclosure of the former in an alternate embodiment. The image processor 102 processes the input into the sensor-stimulator 108, and provides control signals back to the sensor-stimulator 108 so the device can provide processed and output to the optic nerve. That said, in an alternate embodiment, the processing is executed by a component proximate to or integrated with the sensor-stimulator 108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 108 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

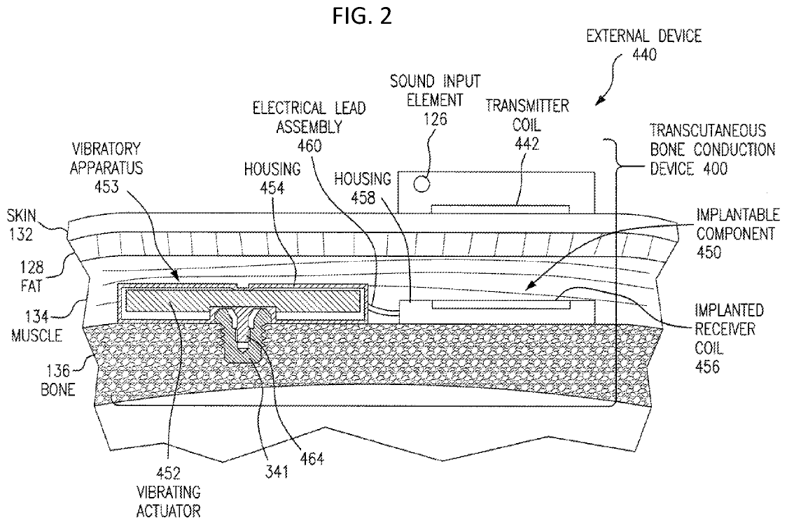

FIG. 2 depicts an exemplary embodiment of a transcutaneous bone conduction device 400 according to an embodiment that includes an external device 440 and an implantable component 450. The transcutaneous bone conduction device 400 of FIG. 2 is an active transcutaneous bone conduction device in that the vibrating electromagnetic actuator 452 is located in the implantable component 450. Specifically, a vibratory element in the form of vibrating electromagnetic actuator 452 is located in housing 454 of the implantable component 450. In an exemplary embodiment, much like the vibrating electromagnetic actuator 342 described above with respect to transcutaneous bone conduction device 300, the vibrating electromagnetic actuator 452 is a device that converts electrical signals into vibration.

External component 440 includes a sound input element 126 that converts sound into electrical signals. Specifically, the transcutaneous bone conduction device 400 provides these electrical signals to vibrating electromagnetic actuator 452, or to a sound processor (not shown) that processes the electrical signals, and then provides those processed signals to the implantable component 450 through the skin of the recipient via a magnetic inductance link. In this regard, a transmitter coil 442 of the external component 440 transmits these signals to implanted receiver coil 456 located in housing 458 of the implantable component 450. Components (not shown) in the housing 458, such as, for example, a signal generator or an implanted sound processor, then generate electrical signals to be delivered to vibrating electromagnetic actuator 452 via electrical lead assembly 460. The vibrating electromagnetic actuator 452 converts the electrical signals into vibrations.

The vibrating electromagnetic actuator 452 is mechanically coupled to the housing 454. Housing 454 and vibrating electromagnetic actuator 452 collectively form a vibratory apparatus 453. The housing 454 is substantially rigidly attached to bone fixture 341.

The implantable component 450 can include a battery or other power storage device, and can be rechargeable.

The embodiments of the implantable components above are examples, and at least some of the various components above can be the same as or correspond to proxies for other implantable devices, such as a middle ear implant or a DACS, etc. The actuator of the device of FIG. 2 can be a proxy for the actuator of a middle ear implant. The coil 456 can be a proxy for the coil of the DACS. Accordingly, any element disclosed herein with respect to one implant can be present in another implant or an analogous component can be therein providing that the art enables such unless otherwise identified.

The examples of implantable devices above are devices that are powered by and/or recharged by a transcutaneous inductance link. Power is transferred from the external component to the implanted component/implantable component via an inductance link. Embodiments include external components/portions thereof that generate an inductance field for powering and/or charging the implant, as will now be detailed.

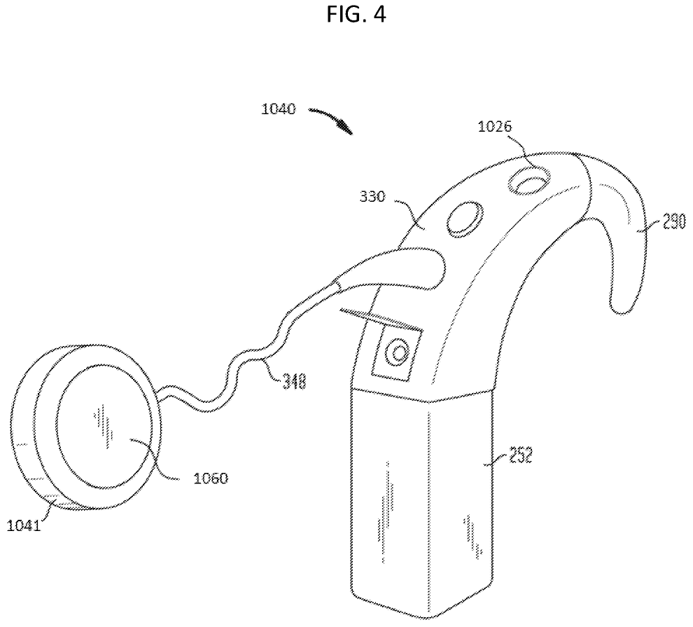

FIG. 4 depicts a cross-sectional view of an exemplary external component 540 corresponding to a device that can be used as external component 142 of FIG. 1 or for example external component 440 in the embodiment of FIG. 4, or any other external component usable with the various prostheses detailed herein. In an exemplary embodiment, external component 540 has all of the functionalities detailed above with respect to external component 142 or external component 440, etc.

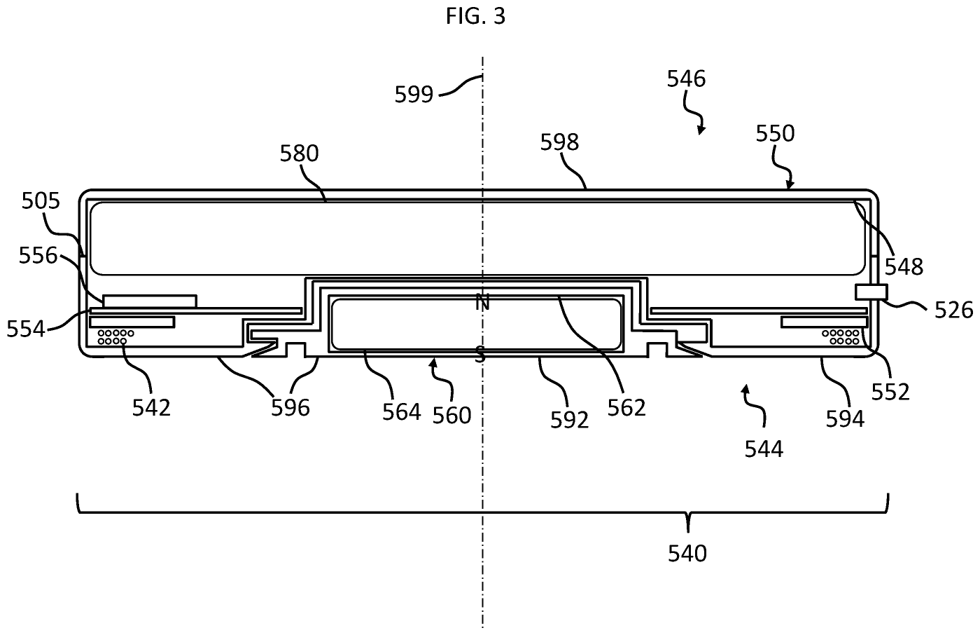

External component 540 comprises a first subcomponent 550 and a second subcomponent 560. It is briefly noted that back lines have been eliminated in some cases for purposes of ease of illustration. It is further noted that unless otherwise stated, the components of FIG. 3 are rotationally symmetric about axis 599, although in other embodiments, such is not necessarily the case.

In an exemplary embodiment, external component 540 is a so called off the ear sound processor. In this regard, in the exemplary embodiment of FIG. 3, the external component 540 includes a sound capture apparatus 526, which can correspond to the sound capture apparatuses 126 detailed above, and also includes a sound processor apparatus 556 which is in signal communication with or located on or otherwise integrated into a printed circuit board 554. Further as can be seen in FIG. 3, an electromagnetic radiation interference shield 554 is interposed between the coil 542 and the PCB 554 and/or the sound processor 556. In an exemplary embodiment, the shield 552 is a ferrite shield. These components are housed in or otherwise supported by subcomponent 550. Subcomponent 550 further houses or otherwise supports RF coil 542. Coil 542 can correspond to the coil 442 detailed above. In an exemplary embodiment, sound captured by the sound capture apparatus 526 is provided to the sound processor 556, which converts the sound into a processed signal which is provided to the RF coil 542. In an exemplary embodiment, the RF coil 542 is an inductance coil. The inductance coil is energized by the signal provided from the processor 556. The energized coil produces an electro-magnetic field that is received by an implanted coil in the implantable component 450, which is utilized by the implanted component 450 as a basis to evoke a hearing percept as detailed above.

The external component 540 further includes a magnet 564 which is housed in subcomponent 560. Subcomponent 560 is removably replaceable to/from subcomponent 550. In the exemplary embodiment of FIG. 3 when utilized in conjunction with the embodiment of FIG. 3, the magnet 564 forms a transcutaneous magnetic link with a ferromagnetic material implanted in the recipient (such as a magnet that is part of the implantable component 450, etc.). This transcutaneous magnetic link holds the external component 540 against the skin of the recipient. In this regard, the external component 550 includes a skin interface side 544, which skin interface side is configured to interface with skin of a recipient, and an opposite side 546 that is opposite the skin interface side 544. That is, when the external component 540 is held against the skin of the recipient via the magnetic link, such as when the external component 540 is held against the skin overlying the mastoid bone where the implantable component is located in or otherwise attached to the mastoid bone, side 546 is what a viewer who is looking at the recipient wearing the external component 540 can see (i.e., in a scenario where the external component 540 is held against the skin over the mastoid bone, and a viewer is looking at the side of the recipient's head, side 546 would be what the viewer sees of the external component 540).

Still with reference to FIG. 3, skin interface side 544 includes skin interface surfaces 592 and 594. Skin interface surface 592 corresponds to the bottom most surface of the subcomponent 560, and skin interface surface 594 corresponds to the bottom most surface of the subcomponent 550. Collectively, these surfaces establish surface assembly 596. Surface assembly 596 corresponds to the skin interface surfaces of the external component 540. It is briefly noted that in some exemplary embodiments, the arrangement of the external component 540 is such that the subcomponent 560 can be placed into the subcomponent 550 such that the bottom surface 592 is recessed relative to the bottom surface 594, and thus the surface 592 may not necessarily contract or otherwise interface with the recipient. It is further briefly noted that in some alternate exemplary embodiments, the arrangement of the external component 540 is reversed, where surface 594 does not contact the recipient because surface 592 remains proud of surface 594 after insertion of the subcomponent 560 into the subcomponent 550.

It is briefly noted that as used herein, the subcomponent 550 is utilized to shorthand for the external component 540. That is, external component 540 exists irrespective of whether the subcomponent 560 is located in the subcomponent 550 or otherwise attached to subcomponent 550.

In the embodiment of FIG. 3 the external component 550 is configured such that the subcomponent 560, and thus the magnet 564 and the housing containing magnet 564 (housing 562), is installable into the external component 540 (i.e., from subcomponent 550) from the skin interface side 544, and thus is installable into the housing 548 at the skin interface side. Also, in some embodiments, the subcomponent 560 is removable from the external component 550. Still with reference to FIG. 3, it can be seen that the external component 540 includes a battery 580. In an exemplary embodiment, the battery 580 powers the sound processor 556 and/or the RF coil 542. As can be seen in FIG. 3, the battery 580 is positioned between the subcomponent 560, and thus the magnet 564, and the side 546 of the external component 540 opposite the side 544 configured to interface with the skin.

FIG. 4 depicts an alternate embodiment of an external component of an external device, BTE device 1040, which can be used in place of external components detailed above, and otherwise has the functionality thereof in at least some exemplary embodiments. More specifically, FIG. 4 depicts a perspective view of a BTE device of a hearing prosthesis. BTE device 1040 includes one or more microphones 1026, and may further include an audio signal under a cover 220 on the spine 330 of BTE device 1040. It is noted that in some other embodiments, one or both of these components (microphone 1026 and/or the jack) may be located on other positions of the BTE device 1040, such as, for example, the side of the spine 330 (as opposed to the back of the spine 330, as depicted in FIG. 4), the ear hook 290, etc. FIG. 4 further depicts battery 252, that is a rechargeable battery, and ear hook 290 removably attached to spine 330.

In an exemplary embodiment, the external component 1040 includes a sound processor or the like located in spine 330. The sound processor is in electronic communication with headpiece 1041 via cable 348. Headpiece 1041 can include an RF coil such as those detailed above. Concomitant with the teachings detailed above with respect to the sound processor of various other embodiments detailed herein, sound captured by the microphone 1026 is transduced into an electrical signal that is supplied to the sound processor, either directly or indirectly. The sound processor processes the signal and converts it into a signal or otherwise processes the signal so as to output a signal via cable 348 to the RF coil located in headpiece 1041, where the RF coil functions according to the teachings detailed above, in at least some exemplary embodiments.

Headpiece 1041 includes a magnet apparatus 351. This magnet apparatus can have the functionality of the subcomponent 550 detailed above.

While the embodiment depicted in FIG. 4 utilizes a cable 348 to establish communication between the spine 330 and the headpiece 1041, in an alternative embodiment, a wireless link is utilized to communicate between the spine 330 and the headpiece 1041.

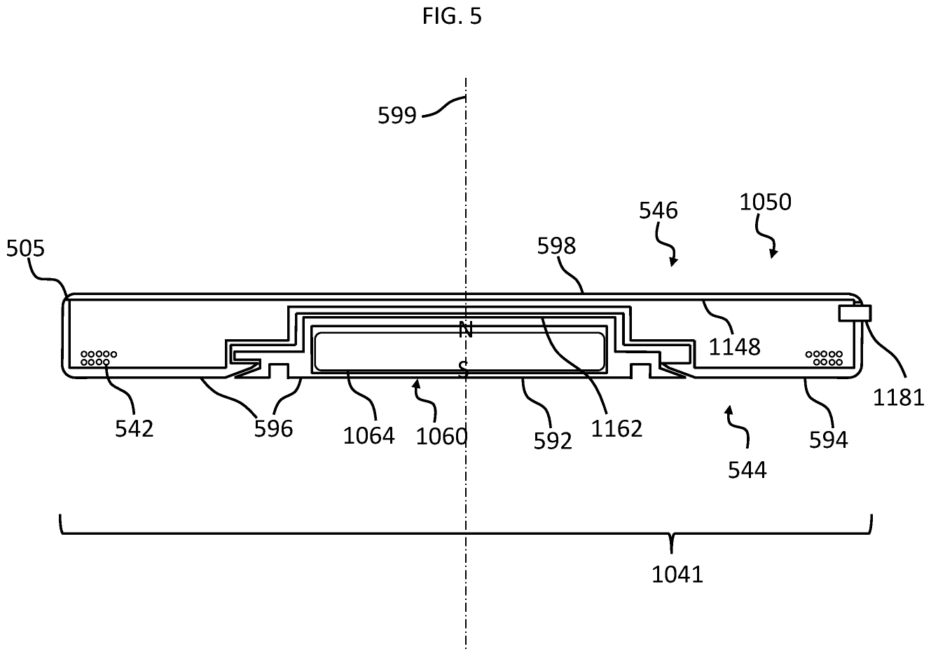

FIG. 5 depicts a cross-sectional view of the headpiece 1041. Here, FIG. 5 is presented with the same frame of reference with respect to FIG. 3 detailed above. Like reference numbers have been utilized in some instances for convenience of conveyance of concept. As can be seen, headpiece 1041 includes a subcomponent 1050 and a subcomponent 1060. In an exemplary embodiment, the subcomponent respectively corresponds, in a conceptual manner, to subcomponents 550 and 560 detailed above. In this regard, subcomponent 550 includes a housing 1148, which contains an RF coil 542. The housing 1148 comprises two sub housings that are joined together at seam 505. Subcomponent 1050 includes cable jack 1181, which is configured to connect the cable 348 to the headpiece 1041.

Subcomponent 1060 includes housing 1162 which contains magnet 1064. In an exemplary embodiment, the functionalities of the components depicted in FIG. 5 can correspond to the functionalities of similar components presented in FIG. 4. In this regard, some of these functionalities will be described in detail below. Briefly, it is noted that the embodiment of FIG. 5 is such that the housing 1148 has a height that is less than the housing 548 of the embodiment of FIG. 4. In the exemplary embodiment depicted in FIG. 5, there is no battery and no sound processor present in headpiece 1041 (because these components can be located in the spine 330, where headpiece 1041 is in signal communication with via cable jack 1181). Thus, the housing can be thinner.

In the embodiment of FIG. 5, the subcomponents interface with one another and are removable and/or attachable with respect to one another in a manner that is the same as or otherwise similar to the embodiment of FIG. 3, where again, additional details of such will be provided below.

In view of the embodiment of FIG. 5, it is to be understood that in an exemplary embodiment, there is a body piece, such as, for example, head piece 1041 (it is noted that in some alternate embodiments, the teachings detailed herein and/or variations thereof can be applicable to components that are not headpieces, but instead, or torso pieces and/or limb pieces etc.) configured for transcutaneous communication with a component implanted in a recipient (e.g., implantable component 450 of FIG. 3, or the implantable component of FIG. 1). In view of FIG. 5, it can be seen that the body piece includes an RF coil 542 and a magnet apparatus in the form of a subcomponent 1060. As can be seen, the RF coil is located on a first side of the body piece relative to an opposite side of the body piece. In this regard, with respect to a plane normal to longitudinal axis 599 bifurcating the geometric body established by the headpiece 1041 (a plane through the geometric center of the headpiece 1041), the RF coil 542 would be located entirely and/or a majority of the RF coil 542 would be located on one side of that plane. Here, the sides of the body piece can be side 544 and 546, the side being opposite to one another. It is further noted that in an exemplary embodiment, with respect to a plane normal to the longitudinal axis 599 bifurcating the center of mass established by the subcomponent 1050 (i.e., without subcomponent 1040 which, owing to the weight of the magnet 1064 would bias the center of mass to one side versus the other a disproportionate amount), the RF coil 542 would be located entirely and/or a majority of the RF coil 542 would be located on one side of the plane. That said, in an alternate embodiment, with respect to a plane normal to the longitudinal axis 599 bifurcating the center of mass established by the entire headpiece 1041 (and also, with respect to the embodiment of FIG. 4 (where external component 550 also corresponds to a body piece), a plane bifurcating center of mass established by the entire external component 540), the RF coil 542 would be located entirely and/or a majority of the RF coil 542 would be located on one side of this plane.

Consistent with the teachings associated with FIG. 3, the embodiment of FIG. 5 is such that the aforementioned first side is a skin interface side (side 544) that consists of a first structure and a second structure. Here, the first structure can correspond to the bottom subcomponent of the housing 1148 and/or 548 (e.g., with respect to the embodiment of FIG. 4, subcomponent 547, which establishes surface 594). Still further, the second structure can be established by the magnet apparatus 1060 (or 560), where the bottom of housing 1162 (corresponding to housing 562 of the magnet apparatus 560) of magnet apparatus 1060 establishes surface 592. In this exemplary embodiment, the first structure established by the housing 1148 houses or otherwise contains the RF coil 542, and the second structure established by housing 1162 houses or otherwise contains the magnet 1064.

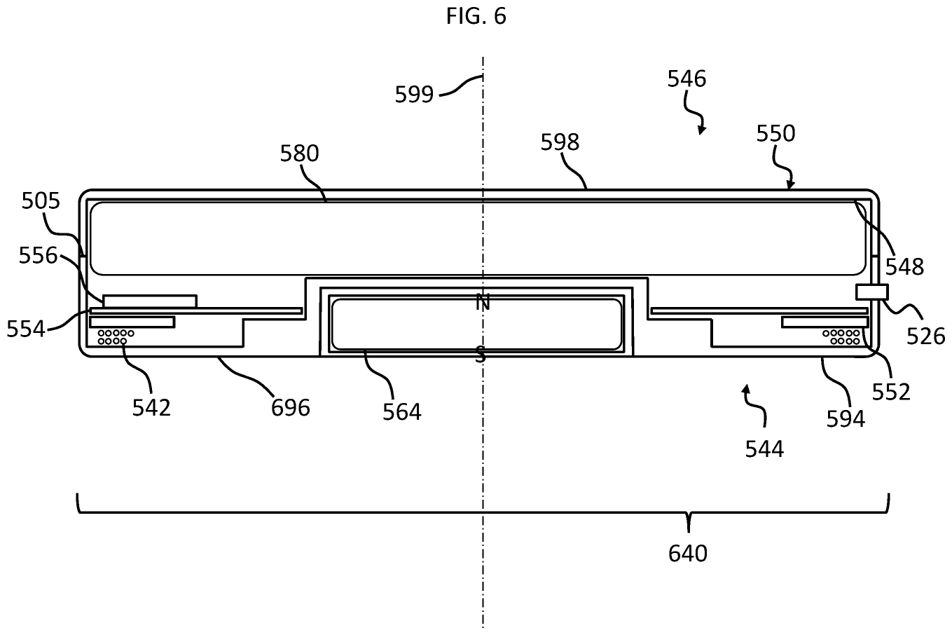

FIG. 6 depicts another exemplary embodiment of an external component 640 that corresponds to the external component 540 above, except that the magnet apparatus is not removable, and in an alternative embodiment, the magnet apparatus is removed from the opposite side 546 (the battery could be a doughnut battery, to enable movement of the magnet apparatus therethrough, or the external component can be configured so that the battery is removable to access the magnet apparatus) as opposed to from the skin interface side 544. This results in a skin interface surface 696 that is without seams and is otherwise uniform and unbroken from side of the external component to the other with respect to the skin interface side 544. In an exemplary embodiment, any one or more of the features of the embodiment of FIG. 6 can be present in the headpiece of the embodiment of FIG. 5 detailed above. In this regard, the skin interface side 544 of the headpiece 1050 can be without seams and otherwise unbroken as is the case with the embodiment of FIG. 5.

The embodiments of FIGS. 1 to 6 are devices that transfer power in some form or another to an implanted device. In some instances, such as the embodiments where the implant is a totally implantable prosthesis, such as a totally implantable hearing prosthesis, the implanted portion includes some form of power storage device, such as a battery, that is rechargeable. The external device can be utilized to charge/recharge that battery utilizing the inductance link from the external component of the implantable component. In this regard, any of the embodiments of FIGS. 1 to 5 can correspond to a generic implant charger in that the external component does not have the sound processor or one or more of the other features detailed above, and instead is directed to solely recharging the implant. The external component can be characterized in at least some exemplary embodiments, as a battery (whether rechargeable or disposable), and an inductance coil that is part of an inductance communication system that is configured to generate an inductance field that can communicate with the implant/transfer power to the implant, and some form of circuitry, which can include logic circuit or control circuit, such as an inductance coil drive circuitry. Some embodiments can include more functional components unrelated to such, but other embodiments can be limited to exactly that (there could be an on-off switch in some such embodiments, and other components, such as a recharge switch (to enable recharging of the battery the external component in some embodiments—in other embodiments, there could be logic that detects when the battery is to be recharged, and thus there may not necessarily be a dedicated recharge switch) but such would be related to the functionality of recharging the implant/transferring power to the implant so that the implant can be recharged—this as opposed to a volume control, or a microphone, which are unrelated to the functionality of recharging the implant or recharging the battery of the external component so that it can be utilized to recharge the implant).

The above said, in some other embodiments, the external component is a device that controls or otherwise provides data (as opposed to simply power) to the implant. It is noted that providing data is not mutually exclusive with providing power. In this regard, in the exemplary embodiment of an external component of a partially implantable hearing prostheses detailed above, such as a partially implantable cochlear implant (non-totally implantable hearing prosthesis), the external component provides power and data/power and control signals that are received by the implant and for all intents and purposes immediately utilized to provide stimulus to the recipient (e.g., to power the cochlear electrode array to provide current to the tissue of the recipient, which current is applied in a controlled manner to evoke the desired hearing percept).

The teachings herein are utilized in at least some embodiments with respect to both types of an external component—the limited external charger and the broader external data source device (which can include the external devices detailed herein that include a sound processor, but also can include by way of example only and not by way of limitation a device that has an external sound processor and a device that simply has microphones or other sound capture devices or other data capture devices, which then provide a signal based there on to the implant, where the implant processes that signal, etc., to evoke the desired hearing percept). It is also noted that the functionality of the two types of external components are not mutually exclusive—an external device can have the functionality of the external sound processors detailed herein, but also can have the functionality of recharging an implanted power storage device.

Power transfer from the external component to the implantable component, such as during recharging of the implantable prosthesis (and thus recharging of the implantable/implanted power storage device—any disclosure herein of recharging implantable prostheses corresponds to a disclosure of recharging the implantable/implanted battery, and vice versa) can result in an increase in temperature of at least some portions of the external component relative to that which would otherwise be the case (e.g., there could be an increase in temperature because the external component is in the sun for example, or because it is hot outside and the recipient just recently moved from inside an air-conditioned environment to an environment that is not air-conditioned (e.g., outside, a factory floor, a warehouse, etc.). In some scenarios, this increase in temperature can be well within comfort and/or safety levels, but in other scenarios, this may not necessarily be the case. The temperature increase can result in a temperature of a skin interface surface, such as skin interface surface 594 and/or skin interface surface 592 and/or the surface assembly 596 and/or the skin interface surface 690, to increase to a level that is uncomfortable and/or unsafe. Hereinafter, these surfaces will be referred to herein as the skin interface surface for linguistic economy. Any reference to such corresponds to a reference to one or more of the aforementioned surfaces unless otherwise noted.

Teachings herein can prevent excessive heating of the external component and/or of the skin interface surface so that the device meets the requirements/guidelines of EN60601-1: “Protection against excessive temperatures and others hazards” which includes some temperature limitation tables applicable to medical equipment that is operated in worst-case normal use including the ambient operating temperature specified in the technical description and/or ISO14708-1/-7, which details that no outer surface of an implantable part of the active implantable medical device shall be greater than 2° C. above the normal surrounding body temperature of 37° C. when implanted, and when the active implantable medical device is in normal operation or in any single-fault condition and/or ISO 14708-3, which details that physical temperature-time limits on heating tissue is given by CEM43, where the temperature of the implanted metal must stay below 43° C.

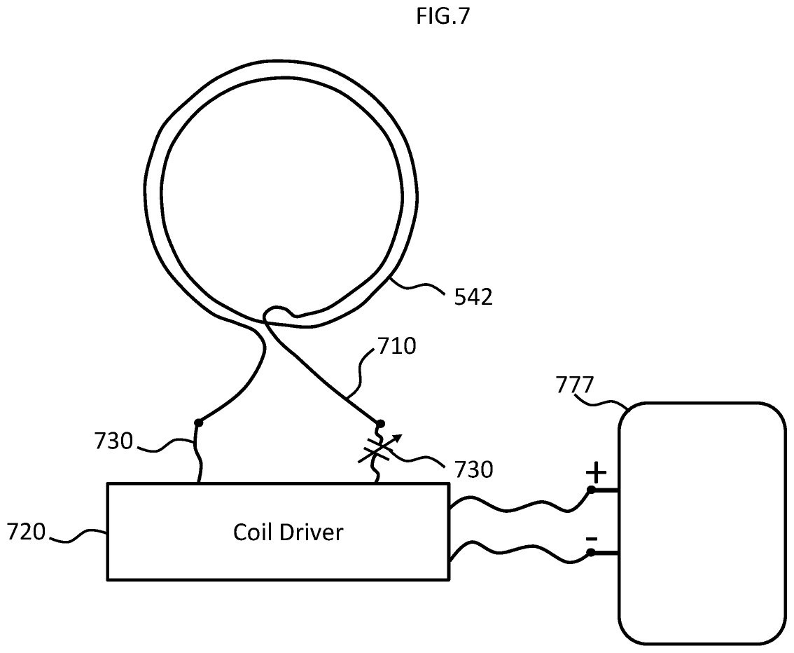

FIG. 7 depicts a high level functional schematic of an inductance recharging system and/or inductance communication system that generates and inductance field to charge and/or otherwise power an implantable component, along with a DC battery 777. The inductance coil 542 can correspond to any of the inductance coil detailed herein, and as can be seen, the coil includes lead portions 710 which are linked to leads 730 of a coil driver 720. In an exemplary embodiment, this coil driver induces an alternating current in the coil 542, and with coil tuning apparatus 730, an inductance field is thus generated and is utilizable to recharge or otherwise power the implant via an inductance link there with. In this regard, FIG. 7 depicts the functional schematic of components of the external devices detailed herein and/or variations thereof.

The coil driver 720 includes circuitry configured to convert DC power from the battery 777 into an alternating current (e.g., by using switching diodes, etc.) that is then applied to the coil 542 to generate the inductance field. The coil driver can include circuitry to vary the inductance field or otherwise vary the amount of current flowing through the coil 542 and/or very the voltage flowing through coil 542 so as to vary or otherwise control an amount of power that is transferred from the external component to the implant (e.g., to reduce a recharge time). In an exemplary embodiment, the driver is a power conversion unit, converting DC current to AC current can utilize one or two or more push-pull switches/transistors. In some embodiments, two half bridges are used to establish a full bridge driver and allow a full AC conversion. This full bridge can be driven by a controller (circuitry configured to do so) which can ensure different switches used are synchronized altogether so that energy wastage is minimized (including prevented). Such device is used for instance on motor drivers, but more recently on wireless chargers.

In an exemplary embodiment, battery 777 corresponds to any one of the batteries detailed above with respect to the external components. The battery 777 can be rechargeable or can be a disposable battery. In an exemplary embodiment, the arrangement of FIG. 7 is embodied in a unitary dedicated external charging device in the form of an off the ear device, such as the device of FIG. 3 or the device of FIG. 6. In an exemplary embodiment, the arrangement of FIG. 7 is embodied in a unitary dedicated external charging device in the form of a BTE device with a headpiece, such as the device of FIG. 4, where battery 252 corresponds to battery 777, and the coil driver is located in the spine 330, and the coils are located in the headpiece 1041. Still, in other embodiments, the arrangement of FIG. 7 can be embodied or otherwise combined with an external sound processor or the like.

Temperature heating of the external component can be a result of a coil and/or driver and/or the battery discharge. Teachings herein can utilize technologies to mitigate the heating effects.

Embodiments can include the utilization of heat pipes, such as ultra-thin and/or flat heat pipes, and/or small thermoelectric coolers (TEC) and miniature fans. Embodiments utilize heat pipes in some instances to extract heat from one side of the external component, such as the skin interface side, and transmit/convey the heat to the other side, thus cooling the one side relative to that which would be the case in the absence of such heat transfer.

An exemplary embodiment utilizes a heat pipe, such as a flat heat pipe, as a charging coil 542. In an exemplary embodiment, the coils can be established by a hose that is made of pure copper or a copper alloy or any other appropriate material that is suitable for utilization in a high-quality inductance coil. This can enable the extraction of heat where it is produced/generated. Indeed, for an implantable device, the heating typically occurs around the implant and the charging coil on the skin. In some embodiments, the coil is less than or equal to 0.25, 0.3, 0.35, 0.4, 0.45, 0.5, 0.6, 0.7, 0.8, 0.9, 1.0, 1.25, 1.5, 1.75, 2, 2.5, 3, 3.5, 4, 4.5, 5, 5.5, 6, 6.5, 7, 8, 9, or 10 mm, or any value or range of values therebetween in 0.01 increments from the skin interface surface. Extracting the heat with the part that transmits the power to the implant can be utilitarian to ensuring or otherwise enabling fast charging, or at least charging within a tolerably short amount of time, because such can mitigate overheating in some embodiments. In an exemplary embodiment, a height of the heat pipe can be less than or equal to 0.5, 0.6, 0.7, 0.8, 0.9, 1.0, 1.1, 1.2, 1.3, 1.4, 1.5, 1.6 or 1.7 mm or any value or range values therebetween in 0.1 mm increments, and width can be less than or equal to 0.5, 0.6, 0.7, 0.8, 0.9, 1.0, 1.1, 1.2, 1.3, 1.4, 1.5, 1.6 or 1.7 mm or any value or range values therebetween in 0.1 mm increments. In an exemplary embodiment might be a Cooler Master Slim heat pipe, or a Petra-flex heat pipe.

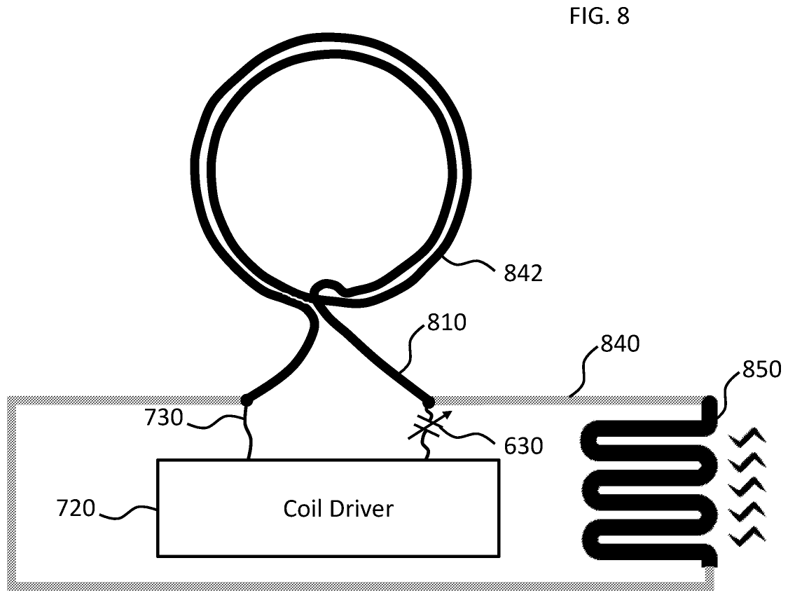

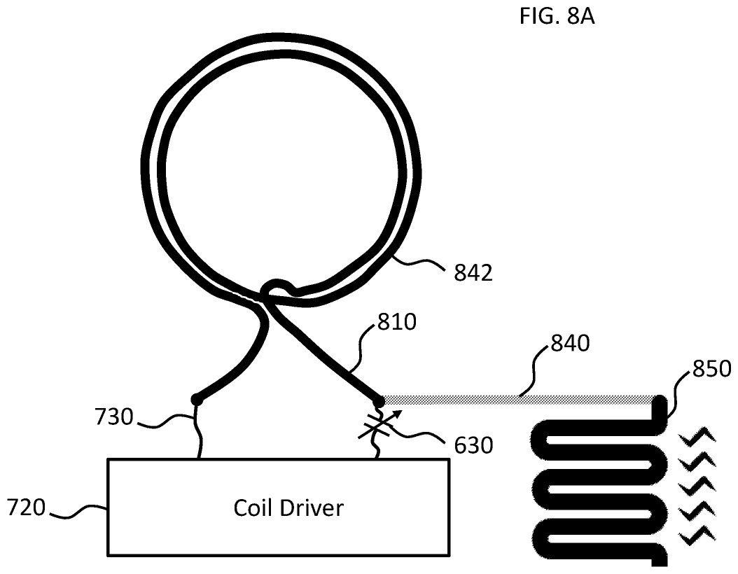

FIG. 8 depicts an exemplary embodiment that utilizes a heat pipe, and in this regard, a flattened heat pipe, as the coil 842 in the leads 810. In this exemplary embodiment, the heat pipe is an electrically conductive heat pipe with respect to the coil portions and the leads portions. The coil driver is electrically connected to the heat pipe via electrical leads 730. Piping 840 is fluidically connected to the leads 810 so as to enable fluid flow from the coil 842 and the leads 810 to the radiator 850 (more on this in a moment). The piping 840 in this exemplary embodiment is electrically nonconductive, thus enabling the coil 842 to be electrically decoupled from the rest of the heat transfer system. An exemplary embodiment, this is not the case—the piping and/or the radiator can also be electrically conductive. In some embodiments, the section of 840 can be a heat pipe in some embodiments, and in other embodiments, it is simply piping to transfer fluid, and thus transfer heat via mass transfer.

The above said, in some embodiments, a portion of the piping can be conductive. Indeed, the heat conduction side of the system can be grounded. By way of example, the radiator 850 can be a ground plane for the inductance system. In an exemplary embodiment, the piping between element 810 and the radiator 850 can be conductive. In an exemplary embodiment, the piping can be the ground plane.

An exemplary embodiment, a tuner cap/tuning cap or the like that is utilized to tune the system is placed “between” (relative to electrical and/or heat transfer paths) the coil 842 and the area where heat is transferred from the coil/the path from a tuning device attaches between the coil and the area where heat is transferred to from the coil. This as opposed to placing a tuner at the other lead 730 of the embodiment of FIG. 8 relative to that which is shown in FIG. 8.

It is briefly noted that while the arrangement shown in FIG. 8 depicts a circuit for fluid to flow around, in an alternate embodiment, there is no true circuit, at least with respect to the view of FIG. 8. In an exemplary embodiment, the fluid might flow back and forth within the heat pipe. By rough analogy, from a distance, a Lionel train on a train platform would travel in a circuit akin to that of FIG. 8 (say a circular train track), whereas a ski lift chair, when viewed from a distance, will by analogy follow the just described “back and forth” path. FIG. 8A depicts such a system.

In any event, in an exemplary embodiment, heat transfer fluid can flow through the piping 840 into the heat pipes and transfer heat generated by the inductance coil into the fluid, and then the fluid can be transported to the radiator 850 where the heat is then radiated out of the system, and then the fluid continues to flow through the circuit back into the coil and the process is repeated.

The radiator 850 can be any type of radiator that can enable the teachings detailed herein. In an exemplary embodiment, a fan with the like can be cold located with or otherwise in communication with the outer surface of the radiator to enhance heat transfer there from. In an exemplary embodiment, the radiator can be a thermoelectric cooler and/or can be in conductive heat transfer communication with a thermoelectric cooler. In an exemplary embodiment, a DC current can be harnessed from the battery 777 directly or indirectly and/or can be obtained from another power source, which DC current can be utilized to achieve a Peltier effect, and thus bring heat from one side of the device to the other.

In another exemplary embodiment, a heatsink can be placed in conductive heat transfer communication with the radiator and/or the radiator can be an expanded heat sink relative to the heat pipe. Any device, system, and/or method that can enhance heat extraction from the heat pipe/fluid flow path can be utilized in at least some exemplary embodiments.

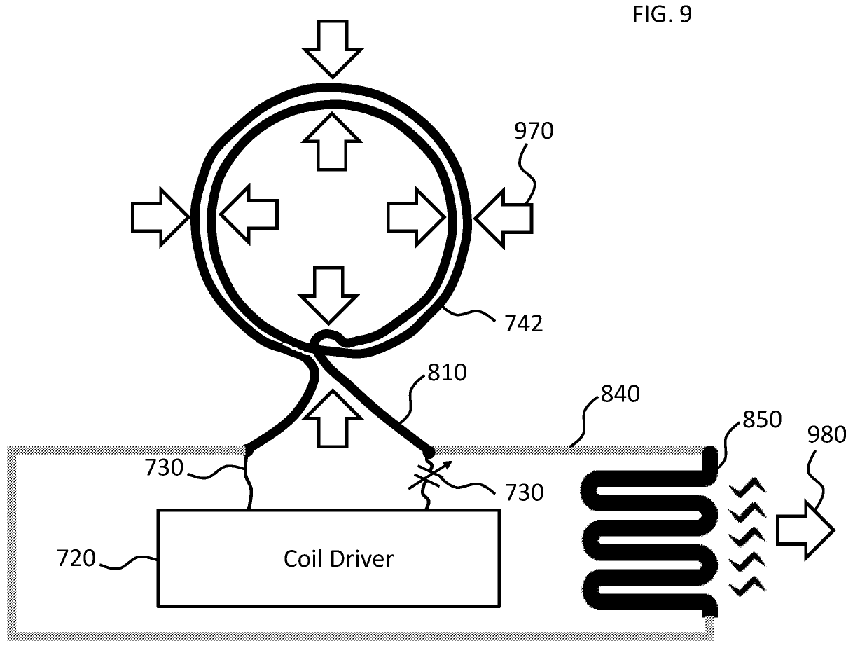

FIG. 9 presents a schematic depicting heat flow into the coil 742, represented by arrows 970 (eight arrows pointing to the coil 742). FIG. 9 also presents a schematic depicting heat flow out of the radiator 850, represented by arrow 980.

In an exemplary embodiment, the coils of the embodiments of FIGS. 1-6 are utilized with the coil of the embodiment of FIG. 8 and/or the arrangement of FIG. 8 is used with the embodiments of FIGS. 1-6. The coil driver can correspond to the circuitry, at least in part, of those embodiments.

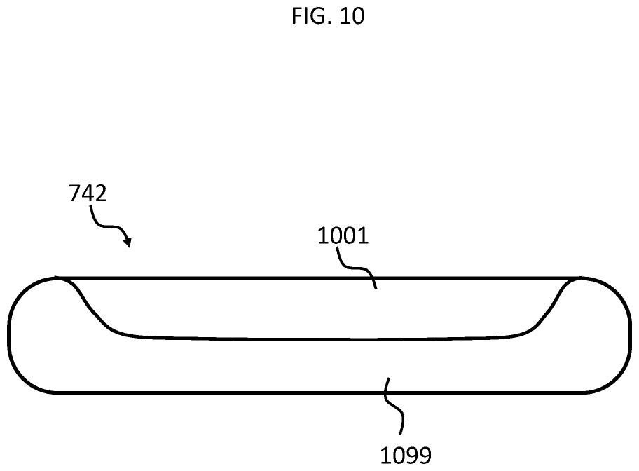

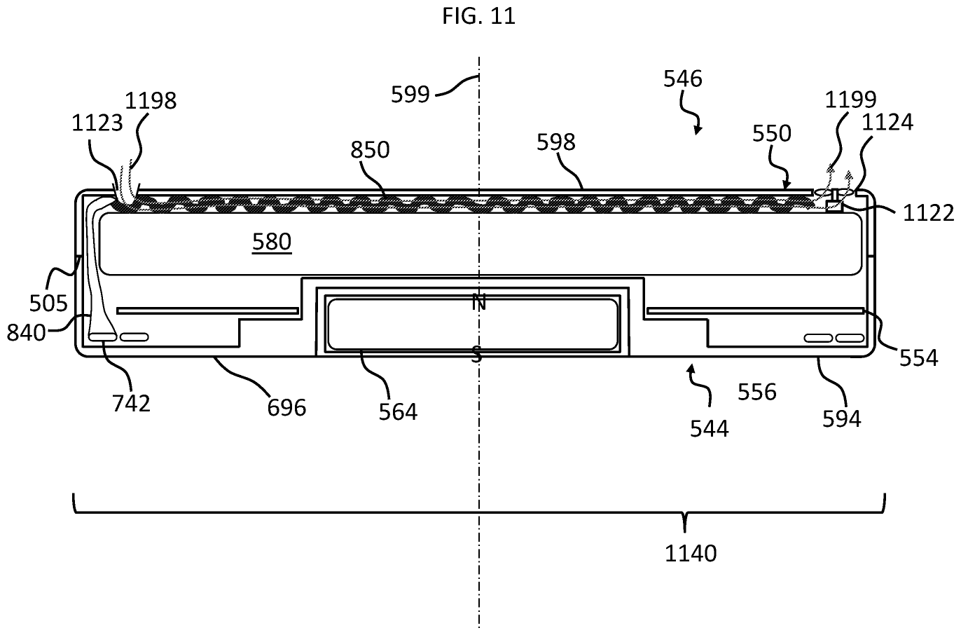

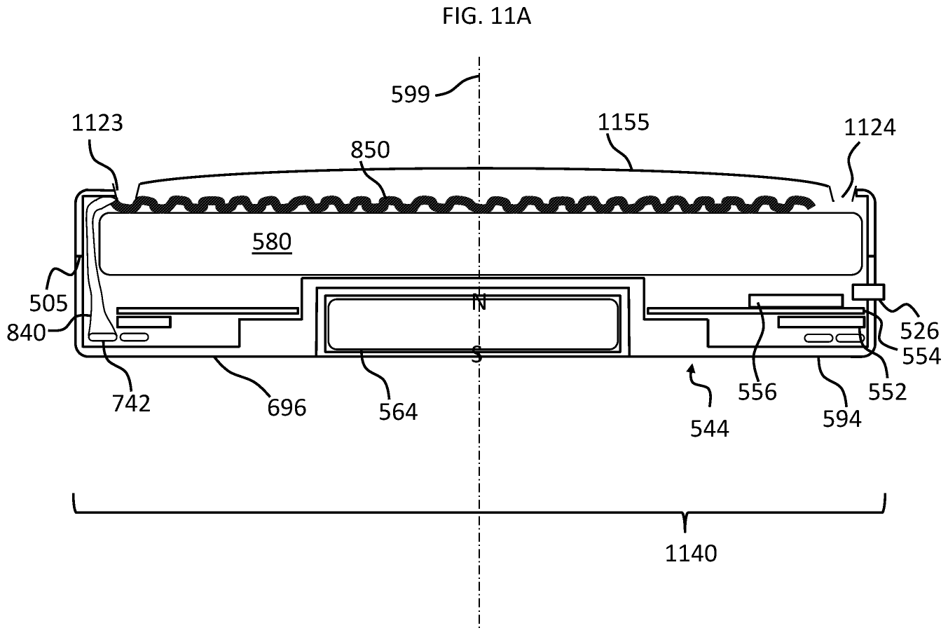

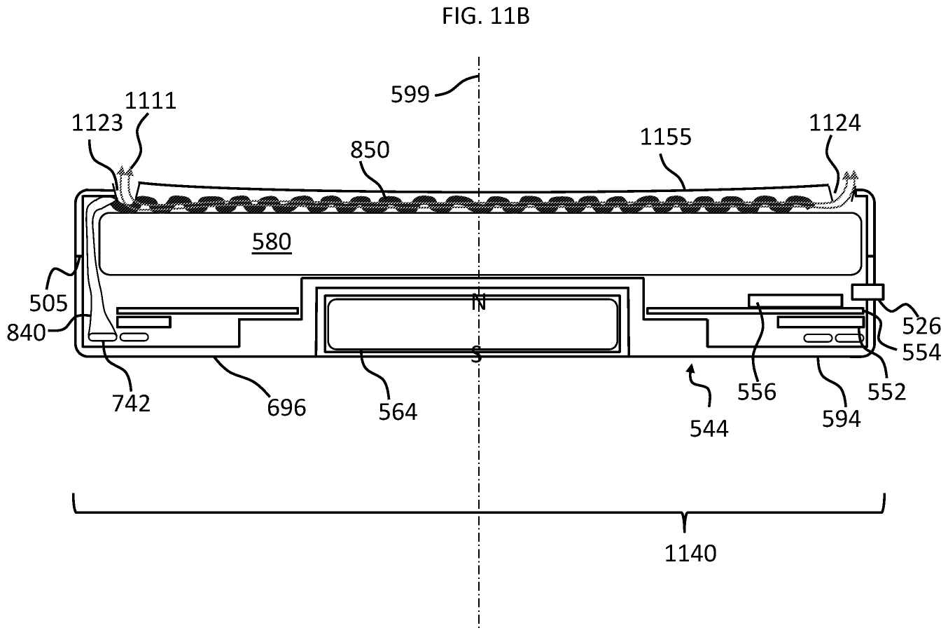

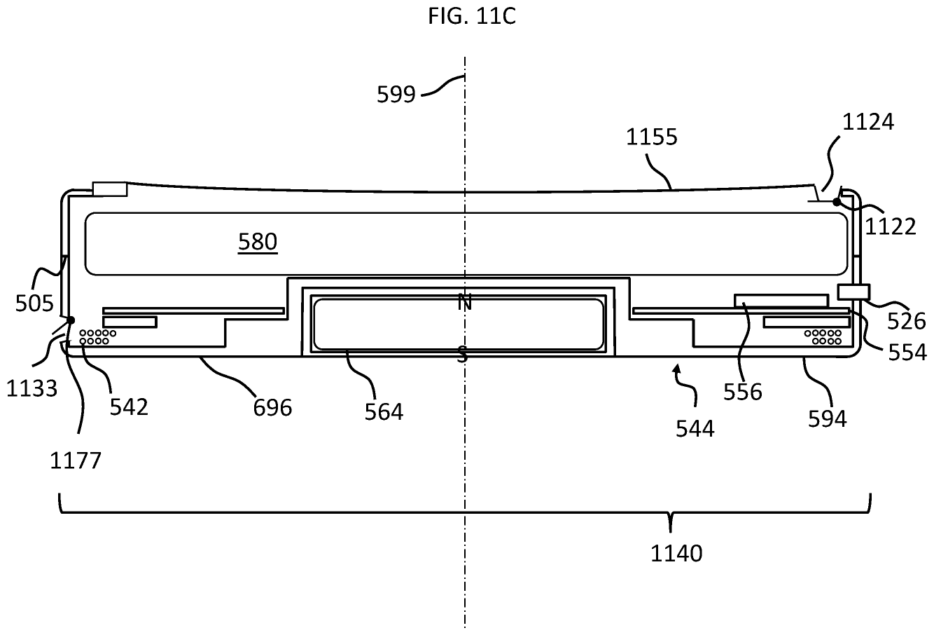

FIG. 10 presents a cross-section of an exemplary flat heat pipe 742. Here, there are two channels, a vapor channel 1001 and a liquid flow channel 1099. These channels are utilized to transfer heat from the area proximate the skin of the recipient to a location elsewhere at the external component. In this regard, FIG. 11 depicts an exemplary external component 1140 that parallels the arrangement of FIG. 6 above. Here, it can be seen that the inductance coil 742 are in the form of heat pipes. In this embodiment, the coil 542 of the embodiment of FIG. 6 has been replaced with the coil 742 that is in the form of a heat pipe. Also seen in FIG. 11 is the piping 840 that is not electrically conductive as detailed above. Piping 840 is depicted as transforming from a flat arrangement to a circular arrangement with distance from the plane of the coil. That said, in an exemplary embodiment, the arrangement of a flat heat pipe can be present with respect to the piping 840. As seen, the piping 840 extends to the radiator 850, which is located away from the skin interface side 544. In this embodiment, the radiator is above the battery 850 but in other embodiments, this can be located on the side of the battery, etc. Any arrangement that will enable the teachings detailed herein can be utilized in at least some exemplary embodiments. As seen, convective airflow is also utilized to enhance heat transfer. Here, there is an intake vent 1123 and an outlet vent 1124. A fan 1122 is located in or proximate the outlet, but that can be located in/proximate the inlet and/or two or more fans can be utilized, one at the intake and one at the outlet. Moreover, the fan can be positioned elsewhere. Any device, system, and/or method that can enable airflow 1198 across the radiator 850 to enhance heat transfer can be utilized in at least some exemplary embodiments. In this embodiment, the fan is powered by the battery 580. In another exemplary embodiment, the fan could be powered by a solar cell or the like. In an exemplary embodiment, a miniature fan can be utilized such as a fan that has a casing that is 15×15×4 mm, which utilizes a current source that is between 2 and 3 Volts (e.g., 2.4 or 2.5 or 2.6 Volts).

Moreover, at least some embodiments do not necessarily utilize an electrically powered fan. In an exemplary embodiment, a manually operated system can be utilized to create a pressure difference to pull or push air across the radiator. By way of example only and not by way of limitation, a diaphragm arrangement can be utilized that will enable a recipient to utilize his or her finger to repeatedly deform the diaphragm and thus create a pressure difference to create airflow across the radiator 850. In this regard, this device can be considered a manual air pump actuatable by a finger. The diaphragm could extend over the radiator between the intake and the outlet—actually, this arrangement would result in both of those components being an intake and an outlet in an alternating manner as the diaphragm is pushed down and then released—pushing down would expel air out of the inside of the housing and thus through the intake an outlet, thus causing them both to be outlets—releasing the diaphragm and having the diaphragm spring back up to its at rest position would then create a lower pressure inside the housing which would then draw air in through the intake and the outlet.

FIGS. 11A and 11B depict an exemplary embodiment of this embodiment. Here, the upper housing wall has been replaced with a diaphragm 1155 that has an at rest position in a bowed out configuration. FIG. 11B depicts the diaphragm 1155 in a depressed position which reduces the volume inside the housing, thus forcing air that is inside the housing out of the housing through the intake 1123 and the outlet 1124 (which at this point should probably be called openings as they both function as an outlet in this embodiment), as represented by arrows 1111. This depressing of the diaphragm 1155 can be achieved by a recipient pressing on the diaphragm with his or her finger. Upon alleviating the force on the diaphragm by removing the recipient's finger for example, the diaphragm 1155 pops back into its at rest configuration as seen in FIG. 11B, thus drawing air into the housing, reversing the directions of the arrows 1111. By repeatedly pressing the diaphragm and allowing the diaphragm to return to its at rest position, airflow into and out of the housing and thus across radiator 850 can be induced beyond that which would otherwise be the case owing to natural principles.

The above said, a manually operated fan could be used. Repeatedly pressing a spring-loaded component for example could cause the fan to turn and thus draw air through the housing and thus across the radiator.

While the embodiments of FIGS. 11-11B have been shown utilizing heat pipes in the radiator 850, in some instances, embodiments that are configured to enable inducement of airflow through the housing/in and out of the housing can be utilized without the heat pipes. FIG. 11C depicts such an exemplary embodiment. Here, there is an intake 1124 and an outlet 1133. The intake 124 is positioned at the top of the housing proximate the diaphragm 1155, and the outlet 1133 is positioned proximate the coils 542. As can be seen, there are valves 1122 and 1177 respectively located at the intake and the outlet. This arrangement works such that when the diaphragm 1155 is press downward, valve 1122 shuts and valve 1133 opens, permitting air to flow from the top of the housing downward past the coils 542 and out the outlet 1133. Conversely, when the diaphragm is released, and the diaphragm springs back towards the at-rest position, valve 1177 become shut and valve 1122 becomes open, permitting air to be sucked in the housing through intake 1124. Repeated actuation of the diaphragm 1155 will induce airflow through the housing and thus create convection cooling of the components therein beyond that which would otherwise be the case. It is noted that this arrangement can be utilized with the aforementioned heat pipes arrangements as well. Any device, system, and/or method disclosed herein can be combined with any other device, system, and/or method providing that such is utilitarian value and the art enables such.

The device can be arranged to automatically provide an indication to a recipient that cooling is needed. This could be an audible beep or could be a voice indication, which could be integrated into the hearing prosthesis arrangement with respect to such embodiments so that the indication results in a hearing percept that is artificially generated by the hearing prosthesis. Upon an indication to the recipient that cooling is needed, the recipient could “pump” air through the housing utilizing the devices just detailed. In this regard, thermocouples or the like or temperature gauges or temperature sensors (one or more) can be located in the housing and/or one components that generate heat, and these sensors can be in signal communication with a processor or otherwise some form of logic circuitry that can enable a determination that a high temperature situation or an increase in temperature situation has occurred that would warrant affirmative action to transfer heat. This determination can be conveyed, or, more accurately, the results of that determination can be conveyed to the recipient or other caregiver so that he or she can engage in the affirmative action of inducing heat transfer in accordance with the teachings detailed herein and/or variations thereof and/or any other regime that would result in affirmative action of transferring heat.

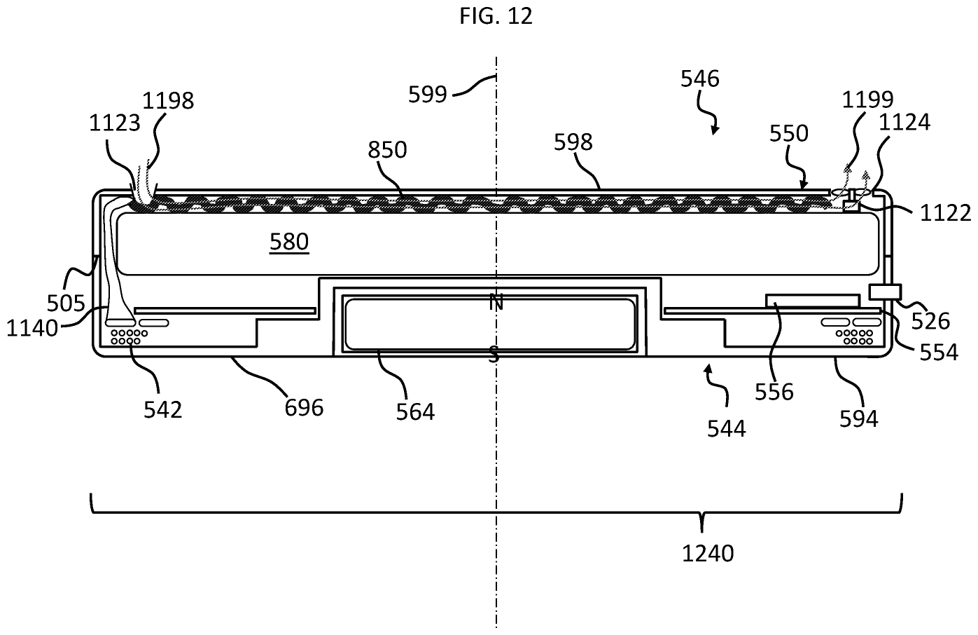

FIG. 12 provides an alternate exemplary embodiment of an external component 1240 where heat pipes 1140 are utilized in conjunction with standard RF induction coils 542. Or, more accurately, there is an external device that utilizes standard RF induction coils 542, and also utilizes a pipe arrangement to transfer heat from the area proximate the skin interface side. In this regard, embodiments are not limited to replacing the standard RF induction coils with heat pipes. The two can be utilized in the same device in some exemplary embodiments.

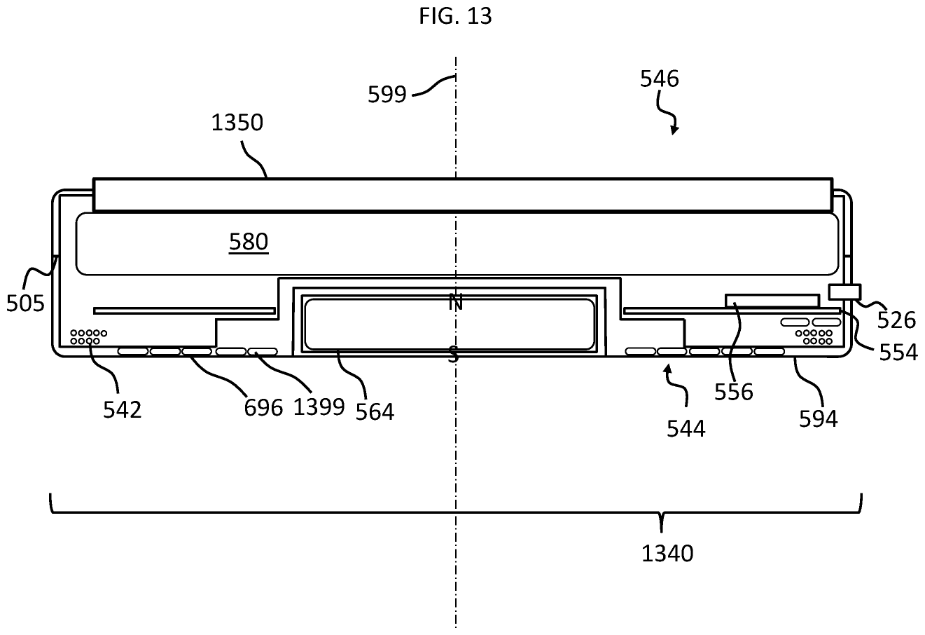

FIG. 13 depicts an exemplary arrangement where heat pipes 1399 are utilized to transfer heat from the skin interface side 544 in general, and the skin interface surface 696 in particular to the opposite side 546 of the external component 1340. Again we see the dual use of heat pipes and traditional standard RF induction coils in the same external component. In this exemplary embodiment, the heat pipes 1399 are in fluid communication with an area proximate a thermoelectric cooler 1350, which can be a Peltier thermoelectric cooling device that is powered by battery 580. The thermoelectric cooler 1350 can have a “cold” side in direct contact with the heat pipes 1399 that extend from the skin interface side 544 up to the opposite side 546 (extensions not shown). This can enhance the heat transfer from the heat pipes 1399, and thus further enhance heat transfer from the skin interface side 544 in general, and otherwise cool the skin interface surface 696 beyond that which would otherwise be the case. In an exemplary embodiment, the thermoelectric coolers that are utilized herein can be, for example, small thermoelectric coolers, and can be single stage 1MD02 thermoelectric coolers.

It is noted that in some embodiments, an electric motor or some other device can be utilized to induce flow within the channels of the heat pipes/increase a flow rate within the channels beyond that which would otherwise be the case.

In view of the above, embodiments include a device, such as an external component of a prosthesis (whether a charging device or an integral component of the prosthesis), comprising an inductive power transmission apparatus, wherein the device includes a dedicated heat transfer arrangement configured to transfer away from the device heat that is generated when transferring power using the device. By “dedicated heat transfer arrangement,” it is meant that there is a recognizable structure in or on the device that one of ordinary skill in the art would recognize is there for the purposes of heat transfer. This as opposed to structure that exists because the device exists, which structure inherently transfers heat. In an exemplary embodiment, the inductance power transmission apparatus is configured to transmit inductance power to a person.

In an exemplary embodiment, the inductive power transmission apparatus includes a resonant tank of a closely coupled inductive link in a wireless power transmission system, and in some exemplary embodiments, the inductive power transmission apparatus includes a heat pipe that is part of a resonant tank of a closely coupled inductive link in a wireless power transmission system. Consistent with the teachings above, in an exemplary embodiment, the inductive power transmission apparatus includes an induction coil that is a heat pipe. Also, as seen above, in an exemplary embodiment, the device is at least part of an external component of a prosthesis system (whether a charger for a totally implantable prosthesis or an external component of a partially implantable prosthesis) that utilizes transcutaneous inductive power transfer to power an implanted component, whether such powering is for direct operation of the implanted component or for recharging of a power storage device that is part of the implanted component.

As noted above, in at least some exemplary embodiments, the external component can be a dedicated charger, while in other embodiments, the external component can be a data transmission device in addition to having the ability to transfer power to the implanted component. Thus, in an exemplary embodiment, the inductive power transmission apparatus can be an inductive communication apparatus.

Also, with reference to the embodiments detailed above that are configured to utilize airflow to enhance heat transfer, in an exemplary embodiment, the device is configured to induce movement of air through the device beyond that which would occur as a result of normal convention to enhance heat transfer from the dedicated heat transfer arrangement.

Consistent with the teachings above, in an exemplary embodiment, there is a device, such as an external component of a prosthesis, which device includes an inductive power transmission sub-system configured to transfer power to an implanted medical device, and a skin interface surface. The device further includes a cooling sub-system configured to cool the skin interface surface. Still further consistent with the teachings detailed above, in some embodiments, the cooling sub-system is integrated with the inductive power transmission sub-system. In this regard, as seen above, in some embodiments, the device is configured to transfer heat with a part that also transmits power, thereby cooling the skin interface surface. Conversely, also consistent with the teachings detailed above, in some embodiments, the cooling sub-system is not integrated with the inductive power transmission subsystem.

In some instances, the device is an off the ear charging device (for example, the embodiment of FIG. 11, which does not include the sound processor components) or an off the ear sound processor (for example, the embodiment of FIG. 11A—it is noted that the embodiment of FIG. 11 can include the sound processor components of FIG. 11A, and the embodiment of FIG. 11A can be a chairing device—some embodiments are such that any component of any embodiment can be combined with another embodiment and any component can be eliminated from an embodiment providing that the art enables such) and the cooling sub-system is configured to transfer heat from the skin interface.

In an exemplary embodiment, any of the devices of FIGS. 3, 5, 6, 11, 11A, 11B, 11C, 12, 13, and 16 can represent an off the ear implant recharger if the sound processor, etc., is removed, if such is present. Note that an off the ear sound processor can also function as an implant recharger. The phrase implant recharger means that it is a dedicated recharger that has no other functionality.

In some embodiments, the device is a behind-the-ear (BTE) device and the skin interface is at a headpiece of the BTE device. In this regard, it is noted that behind the ear devices can be dedicated chargers, where the ear is utilized to support the battery and other components instead of utilizing the transcutaneous magnetic link to support the battery. This can, in at least some embodiments, enable the use of a larger and/or heavier battery relative to that which would otherwise be the case. In this embodiment, the BTE device simply has the sole functionality of charging the implant. Still, in other embodiments, the BTE device can be a sound processor/have the functionality of a sound processor. In both arrangements, there can be utilitarian value with respect to utilizing a cooling subsystem. With respect to embodiments that utilize a BTE device, the heat pipes can extend from the headpiece to the spine of the BTE device. The heat pipes can extend through cable 348, and a heat exchanger can be located in the spine 330. The heat pipes can enable the flow of cooling fluid from the headpiece to the heat exchanger and then back to the headpiece in a manner analogous to the operations detailed above. In an alternate embodiment, the cable 348 can be a heat exchange device. The cable could be ribbed or can include ribbed sections that would enhance heat transfer radiation and/or convection beyond that which would be the case with respect to a cylindrically shaped smooth cable.

It is also noted that in some embodiments, there can be a scenario where the body proper (spine, battery and/or ear hook) of the BTE device could experience a higher than desired temperature. In this regard, embodiments include a BTE device where the heat transfer arrangements and/or cooling arrangements herein are implemented in the BTE device body and/or are otherwise utilized to reduce a temperature of a surface of the BTE device body that contact skin relative to that which would otherwise be the case without such implementations. By way of example, the battery 252 could become heated during discharge (or charging—more on this in a moment) and/or the coil driver could produce heat, which coil driver is located in the spine 330, or any other component located in the BTE device body could produce heat, and thus there could be utilitarian value with respect to cooling the skin interfacing services. In an exemplary embodiment, heat pipes can be located proximate the outer surface of the battery 252 and/or the outer surface of the spine 330 and/or the outer surface of the ear of 290, which outer surface contacts the skin during normal use.

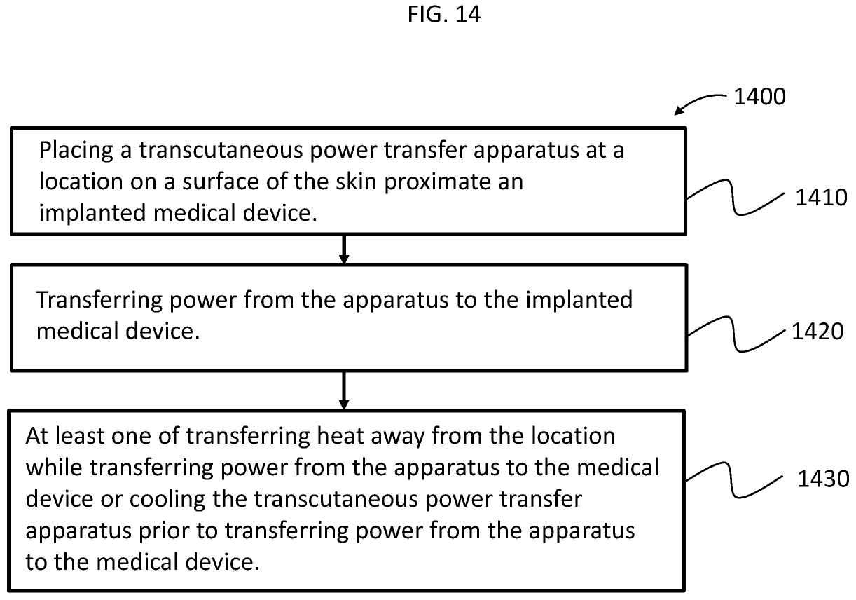

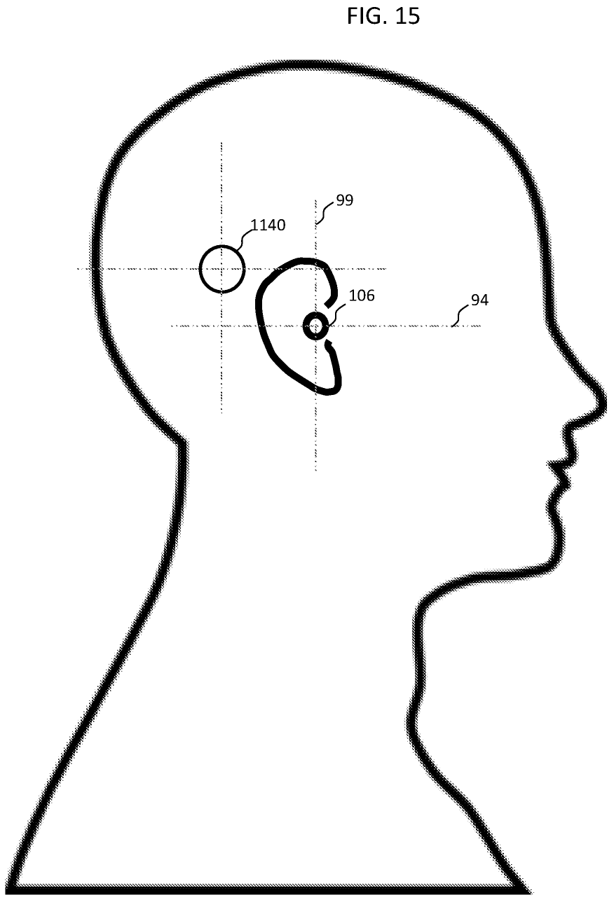

Embodiments also include methods. For example, FIG. 14 presents an exemplary flowchart for an exemplary method, method 1400. Method 1400 includes method action 1410, which includes placing a transcutaneous power transfer apparatus (e.g., the external components detailed herein, whether dedicated power recharging device or a data communication device that also transfers power) at a location on a surface of the skin proximate an implanted medical device. This can be at a location off an ear of the recipient. This can correspond to placing the headpiece (e.g., 1140) at the location shown in FIG. 15, which should be considered to scale vis-á-vis a human factors engineering 50 percentile male or female of 40 years of age born in the United States of America as of Aug. 26, 2020. FIG. 15 depicts an exemplary placement of the external component 1140 against the head of a recipient from the frame of reference of the viewer looking at a right side of a recipient, where the recipient is looking ahead (the “right side” being the recipient's right side—the side of the recipient's right hand. Shown in FIG. 15 for purposes of reference is the pinna of the recipient, and the ear canal of the recipient 106. Horizontal axis 94 and vertical axis 99 are centered at the center of the outer opening of the ear canal 106. Horizontal axis 94 corresponds to the gravitational horizon, and vertical axis 99 is parallel to the direction of gravity. A distance in the X axis and/or the Y axis from the center locations of the canal 106 and the external component 140 can be any of 2, 2.25, 2.5, 2.75, 3, 3.25, 3.5, 3.75, 4, 4.25, 4.5, 4.75 or 5 inches or more or any value or range of values therebetween in 0.01 inch increments.

In the embodiment discussed, the action of placing the headpiece against the skin of the recipient results in an inductance coil of the headpiece being effectively centered with an implanted inductance coil implanted underneath the skin of the recipient. The separation between the two coils can be less than, greater than or equal to 2, 3, 4, 5, 6, 7, 8, 9, 10, 11, 12, 13, 14, 15, 16, 17, 18, 19, or 20 mm or more, or any value or range of values therebetween in 0.1 mm increments.

Method 1400 further includes method action 1420 which includes transferring power from the apparatus to the implanted medical device. In this embodiment, this is done via the inductance link established by the external component and the implanted component. This can correspond to transferring only power to the implanted device or the transfer of both power and data to the implanted device, the latter being what happens in a partially implantable cochlear implant for example.

Method 1400 also includes method action 1430, which includes at least one of transferring heat away from the location while transferring power from the apparatus to the medical device or cooling the transcutaneous power transfer apparatus prior to transferring power from the apparatus to the medical device. With respect to the former, such can be enabled utilizing the teachings detailed above, such as for example, utilizing a cooling subsystem, such as one of those detailed above by way of example. With respect to the latter feature, additional details of this will be described below. But briefly, there can be utilitarian value with reducing a temperature of the external component relative to that which would otherwise be the case in the absence of affirmatively cooling the apparatus priority utilization of such so that the total heat energy released will ultimately result in a lower overall temperature of the skin interface surface relative to that which would otherwise be the case all other things being equal, and thus effectively achieving an analogous utilitarian result to that which is achieved utilizing the cooling subsystems detailed herein. More on this below. We first focus on the action of transferring heat away from the location.

In an exemplary embodiment, the action of transferring heat away from the location is executed by moving a fluid from a location inside the apparatus and proximate a surface of the apparatus that interfaces with the surface of the skin to a location away from that location inside the apparatus. This can be achieved by way of example utilizing the pipes of the embodiment of FIG. 11 detailed above. It is noted that the movements of the fluid within the pipes can be a result of convection currents and/or can be induced via the utilization of a device that creates a pressure differential within the heat pipes. This can also be achieved by way of example utilizing the arrangement of FIG. 11C, where ducting extends from the intake 1124 to a location proximate the skin interface side 544 such that the airflow flows over the inside surface of the wall that establishes the skin interface surface 690 before the airflow exits the outlet 1133. It is noted that fins or other heat transfer enhancing devices can be located on the inside surface of the wall that establishes the skin interface surface 690 to enhance the cooling.

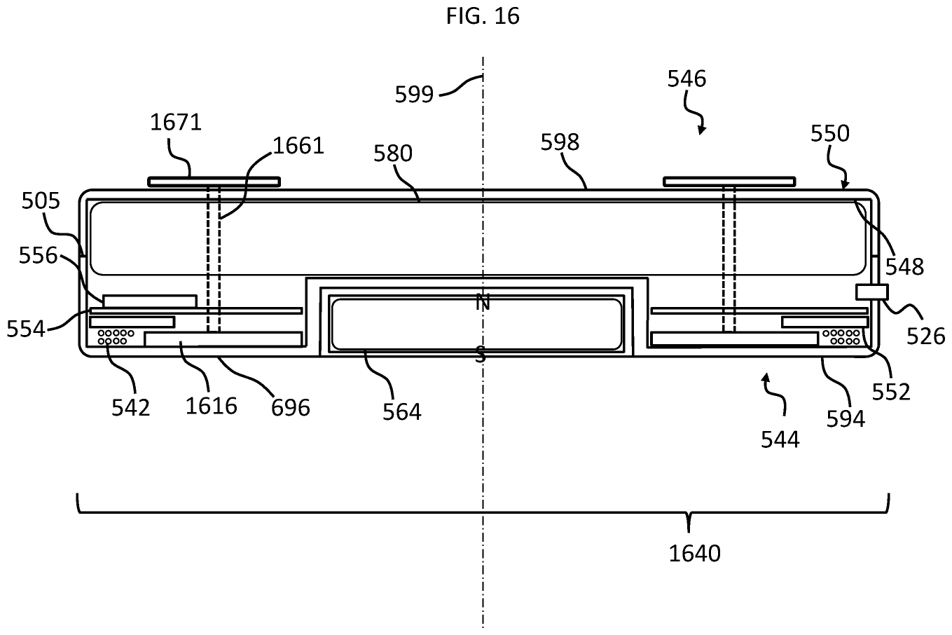

Consistent with the teachings above, in an exemplary embodiment, method action 1430 is executed using thermoelectric cooling. As can be achieved by the arrangement of FIG. 13 as seen above. This can be achieved also by the arrangement seen in FIG. 16. In this regard, FIG. 16 presents an exemplary external component 1640. As can be seen, thermoelectric cooling devices 1616 are located on the bottom wall of the housing, which bottom wall establishes the skin interface surface 696. These devices, which can be Peltier devices 1616, have the “cold side” facing the bottom wall, and the hot side facing away. These can be powered by DC current from the battery 580. Shown in dashed lines for ease of clarification are heat sinks 1661. These are connected to heat sink 1671, which corresponds to a radiator device. In this exemplary embodiment, heat sinks 1661 can extend through the battery or can extend around the battery to reach the opposite side 546. The housing facing side of heat sinks 1671 can be above the top surface of the housing to enable airflow between these two components to increase the transfer therefrom. Ribs or other devices that increase heat transfer can be located on the outside of the housing of the external component 1640 on the opposite side 546.

It is noted that in an alternate embodiment, the heat sinks 1671 can additionally or instead extend about the outer periphery of the external component 1640 (this could be a band that extends concentrically about the axis 599), which component is in heat transfer communication with the Peltier devices 1616 via heat sinks.

It is also noted that air can be blown across the hot sides of the Peltier devices 1616 so as to transfer heat from those devices.

Of course, method action 1430 can be executed using heat pipe(s).

In an exemplary embodiment, method action 1420 is executed during an action of fast charge of an implanted prosthesis having an implanted power storage device. This is distinguished from normal charging. Because of the rapid discharge of the battery that would result and/or because of the higher load on the coil driver, the temperature of the external component would increase relative to that which would be the case if a less rapid charging regime was utilized.

An exemplary embodiment of an exemplary method further includes the action of charging the implanted prostheses during a non-fast charge action. During this action, the action of transferring heat away from the location is not executed during the non-fast charge action. In this regard, in an exemplary embodiment, the teachings herein can be applied in a controlled or otherwise limited manner when “necessary” (very loosely termed with respect to some embodiments or otherwise scenarios) and otherwise not utilized when not necessary. Corollary to this is that in at least some exemplary embodiments, the action of transferring heat away from the location (method action 1430) is automatically executed in response to a determination that a variable indicative of skin temperature and/or a rate of change of skin temperature has changed by a predetermined amount. By way of example only and not by way of limitation, this can correspond to the utilization of a thermocouple or otherwise a temperature sensor at or proximate the skin interface surface 690. The predetermined amount can be any amount they can have utilitarian value. The variable can be a latent variable as well. Indeed, the aforementioned sensors proximate the skin interface surface could be embedded inside the bottom housing wall and spaced away from the skin of the recipient. Thus, data therefrom would not necessarily correlate to the temperature of the skin. Still further, a temperature of the inside surface of that wall can also be measured, and based on calculative and/or empirical and/or modeled data, an estimate as to the skin temperature can be obtained. Any device, system, and/or method that can enable the teachings detailed herein vis-à-vis determining skin temperature features can be utilized in at least some exemplary embodiments providing that the art enable such.

The teachings detailed herein can enable the utilization of charging techniques and/or power techniques in relatively high ambient environment temperatures, such as for example, during a heat wave in the south east or south west United States, where a recipient of the prosthesis is outside, and at least outside for an extended period of time. In an exemplary embodiment, the methods and devices and systems are used in an ambient environment in the shade or in sunlight after a recipient and/or the external device has been in that environment for at least 1, 1.5, 2, 2.5, 3, 3.5, 4, 4.5, 5, 5.5, 6, 7, or 8 hours prior to commencement of power transfer, where the ambient environment temperature is above 35, 36, 37, 38, 39, 40, 41, 42, 43, 44, or 45 degrees Celsius at the time that power transfer is commenced and has been such for at least 0.5, 1, 1.5, 2, 2.5, 3, 3.5, 4, 4.5, 5, 5.5, 6, 7, or 8 hours, prior thereto.

In this regard, a temperature ambient the location is above any of the aforementioned temperatures for at least 0.5 1, 1.5, 2, 2.5, 3, 3.5, 4, 4.5, 5, 5.5, 6, 7, or 8 hours, before commencement of the action of charging and the temperature at the location is maintained at below 41 degrees Celsius during the entire time of transferring power from the apparatus to the medical device, which time is at least 0.1, 0.2, 0.3, 0.4, 0.5, 0.6, 0.7, 0.8, 0.9, 1, 1.25, 1.5, 1.75, 2, 2.25, 2.5, 2.75, 3, 3.25, or 3.5 hours, or any value or range of values therebetween in 0.01 hour increments.

Some exemplary embodiments include executing one or more of the implant charging actions detailed herein while maintaining a skin temperature at the location of power transfer below 43, 42, 41, 40, 39, 39, 37, 36 or 35 degrees Celsius for the entire time that charging is executed. In an exemplary embodiment, any one or more the method actions detailed herein begins with a skin temperature, prior to contact of the external device to the skin of the recipient, that is less than greater than or equal to 29, 30, 31, 32, 33, 34, 35, 36, 37 or 38 degrees Celsius or any value or range values therebetween in 0.1° increments. Starting from this skin temperature, the method actions detailed herein can be executed such that the implant is recharged while maintaining the skin temperature at the aforementioned temperatures at the beginning of this paragraph.

In an exemplary embodiment, the teachings detailed herein can be utilized to increase a charging time/increase the amount of time that an external component can be utilized to charge an implanted component/implanted battery. In this regard, in an exemplary embodiment, utilizing a device without the affirmative heat transfer actions detailed herein might result in a temperature of skin of the recipient reaching 39, 40, 41, 42 or 43 degrees Celsius or more during the charging operation. These temperatures can be dangerous and/or otherwise uncomfortable. The recipient of the prostheses that is being charged might be inclined to stop the charging process because the heating of the skin is uncomfortable.

That said, in some embodiments, the device may automatically shut off or otherwise stop charging or otherwise reduce the rate of charging because the device senses that the skin temperature is being raised to a unacceptable and/or undesirable level (either by a direct skin temperature sensor that is part of the external component were utilizing a latent variable to detect such, such as that which may be the case utilizing a sensor that detects the temperature of a portion of the external component and extrapolates or otherwise deduces an estimated temperature of the skin). Accordingly, in at least some exemplary embodiments, by utilizing the heat transfer teachings detailed herein, that skin temperature will not reach these unacceptable or otherwise uncomfortable temperatures, and thus the external component can be used for a longer period of time to charge the implant (while maintaining the charging rate that existed, say, for at least 5, 6, 7, 8, 9, 10, 11, 12, 13, 14, 15, 20, 25, 30, 35, 40, 45, 50, 55 or 60 minutes or more during the charging period—as opposed to reducing the rate to reduce the temperature), thus enabling the implant to be charged “more” and/or more quickly than that which would otherwise be the case, all other things being equal.

Indeed, in an exemplary embodiment, the actions of charging the implant are executed such that during a time of recharging, a rate of charging does not deviate more than 2, 3, 4, 5, 6, 7, 8, 9, 10, 15, 20, 25 or 30% from an average rate of charge of a charging process (mean, median and/or mode), excluding ramp up or ramp down periods that are utilized for battery life preservation, etc., for more than 5, 10, 15, 20, 25 or 30% of the total time that the external device is against the skin of the recipient. Accordingly, in an exemplary embodiment, there are methods of charging where the transmitted power is not purposely powered down or otherwise limited for temperature reasons (it might be limited for other reasons).

In an exemplary embodiment, the methods detailed herein can be executed 10, 15, 20, 25, 30, 35, 40, 50, 60, 70, 80, 90, 100, 125 or 150 times or more with the same device(s) while meeting the parameters detailed herein.

As noted above, there can be utilitarian value with respect to cooling the headpiece at least of the external component that is utilized to charge the implanted component relative to that which would otherwise be the case for commencing charging of the implanted device. Here, in this embodiment, the external component would start off at a lower temperature relative to that which would otherwise be the case owing to ambient conditions, and thus the heat generated as a result of the recharging of the external component would be “taken up” by the fact that the external component starts off colder than that which would otherwise be the case.

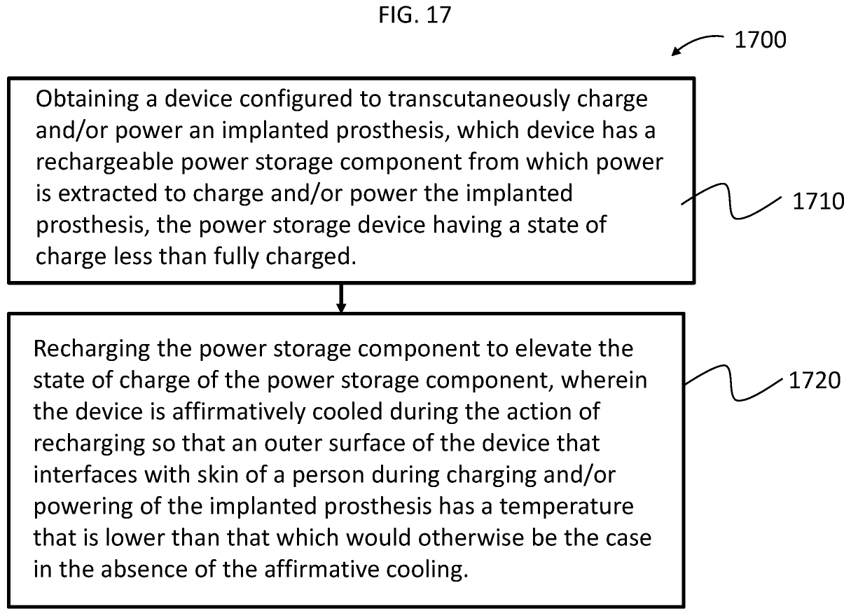

FIG. 17 presents a flowchart for another exemplary method, method 1700, according to an exemplary embodiment. Method 1700 includes method action 1710, which includes obtaining a device configured to transcutaneously charge and/or power an implanted prosthesis, which device has a rechargeable power storage component from which power is extracted to charge and/or power the implanted prosthesis, the power storage device having a state of charge less than fully charged. In an exemplary embodiment, this can be any of the external devices detailed above that can enable this method to be practiced. The device can be the external device of FIG. 1 or FIG. 6 or FIG. 11, by way of example only.

Method 1700 includes method action 1720, which includes recharging the power storage component to elevate the state of charge of the power storage component, wherein the device is affirmatively cooled during the action of recharging so that an outer surface of the device that interfaces with skin of a person during charging and/or powering of the implanted prosthesis has a temperature that is lower than that which would otherwise be the case in the absence of the affirmative cooling.

Here, there can be utilitarian value with respect to cooling or otherwise preventing a temperature of the external component from reaching a certain level relative to that which would otherwise be the case in the absence of the implementations of the teachings detailed herein/reducing the temperature of the external component relative to that which would otherwise be the case, so that when the external component is placed against the skin of the recipient to recharge the implanted prostheses, the temperature the device is lower than that which would otherwise be the case, which could result in a more safe utilization of the component and/or a more comfortable utilization of the component relative to that which would otherwise be the case. In an exemplary scenario of use, the recipient has an external device that has lost its charge or otherwise is not sufficient to power otherwise provide power to the implant, and the recipient needs at least a partially charged external component. The recipient also needs this sooner rather than later. Accordingly, for example, a fast charge is utilized to charge the external device, which could be a charger of a fully implantable hearing prostheses. The fast charge would potentially elevate the temperature of the external component, and could potentially elevate the temperature of the external component to a level that is unsafe or at least would otherwise be uncomfortable when used. The implementation of method action 1720 thus limits the amount of temperature increase.

Accordingly, there can be utilitarian value with respect to utilizing the teachings detailed herein so as to charge an external device in a relatively faster manner than that which would otherwise be the case so that that external device can be utilized in short order without having to wait for the device to cool down before placing against the skin of the recipient.

Accordingly, in an exemplary embodiment, the recharging of method action 1720 is executed such that the state of charge of the battery of the obtained component is increased by at least or equal to 10, 15, 20, 25, 30, 35, 40, 45, 50, 55, 60, 65, 70, 75, 80, 85, 90, 95 or 100 percent or any value or range of values therebetween in 1% increments within a time period no longer than or equal to 0.1, 0.2, 0.3, 0.4, 0.5, 0.6, 0.7, 0.8, 0.9, 1, 1.25, 1.5, 1.75, 2, 2.25, 2.5, 2.75, 3, 3.25 or 3.5 hours or any value or range of values therebetween in 0.01 hour increments, which battery has a new rating of greater than or equal to 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500 milli-Amp hours or more or any value or range of values therebetween in 1 milli-Amp hour increments, inclusive (e.g., 265, 444, 111 to 33). In some embodiments, the range is from 50 to 250, or 70 to 225, or 90 to 200 mAH, and any avalue or range of values therebetween in 1 mAH increments.

In an exemplary embodiment, the devices and systems herein are such, and the methods are such that power can be transferred/is transferred at a rate of at least and/or equal to 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 10, 11, 12, 13, 14, 15, 16, 17, 18, 19, 20, 21, 22, 23, 24, 25, 26, 27, 28, 29 or 30 or more times or any value or range of values therebetween in 0.05 increments for at least or equal to 30, 35, 40, 45, 50, 55, 60, 65, 70, 75, 80, 85, 86, 87, 88, 89, 90, 91, 92, 93, 94, 95, 96, 97, 98, 99 or 100% of the charging time or any value or range of values therebetween in 1% increments, times which would be the case in the absence of the cooling arrangements herein while maintaining the same temperature of the skin interface of the device and/or preventing the temperature from exceeding 35, 36, 37, 38, 39, 40 or 41 degrees C. where an ambient air temperature has been within 10 degrees of such temperature, all other things being equal. In an exemplary embodiment, there are batteries that are used with the prostheses herein that have a standard charging rate of XYZ mAh. In some embodiments, charging can take place at a rate of 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 10, 11, 12, 13, 14, 15, 16, 17, 18, 19, 20, 21, 22, 23, 24, 25, 26, 27, 28, 29 or 30 or more times or any value or range of values therebetween in 0.05 increments times XYZ, for at least or equal to 30, 35, 40, 45, 50, 55, 60, 65, 70, 75, 80, 85, 86, 87, 88, 89, 90, 91, 92, 93, 94, 95, 96, 97, 98, 99 or 100% of the charging time or any value or range of values therebetween in 1% increments.

In an exemplary embodiment, a maximum and/or average outer diameter of the inductance coil of the external device is less than or equal to 15, 16, 17, 18, 19, 20, 21, 22, 23, 24, 25, 26, 27, 28, 29, 30, 31, 32, 33, 34, or 35 mm or any value or range of values therebetween in 0.1 mm increments. The wire diameter of an individual strand can be that which is standard for cochlear implants. The coil arrangement can be that which is used on the Nucleus 7™ as of Aug. 26, 2020, as is approved for sale and is being sold in the United States of America.

In an exemplary embodiment, the devices and systems herein are such, and the methods are such that power can be transferred/is transferred at a rate of at least and/or equal to 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500 milli-amps per hour while achieving the performance goals herein.

In some embodiments, the outermost diameter of the head piece is 2, 3, 4, 5, 6, 7, 8, 9, 10, 11, 12, 13, 14, or 15 mm or any value or range of values therebetween in 0.1 mm increments larger than the outermost diameter of the coil. In some embodiments, a total height of the headpiece is less than or equal to 15, 16, 17, 18, 19, 20, 21, 22, 23, 24, 25, 26, 27, 28, 29 or 30 mm or any value or range of values therebetween in 0.1 mm increments. In some embodiments, a weight of the headpiece, including the battery, is less than or equal to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 grams or any value or range of values therebetween in 0.1 gram increments.

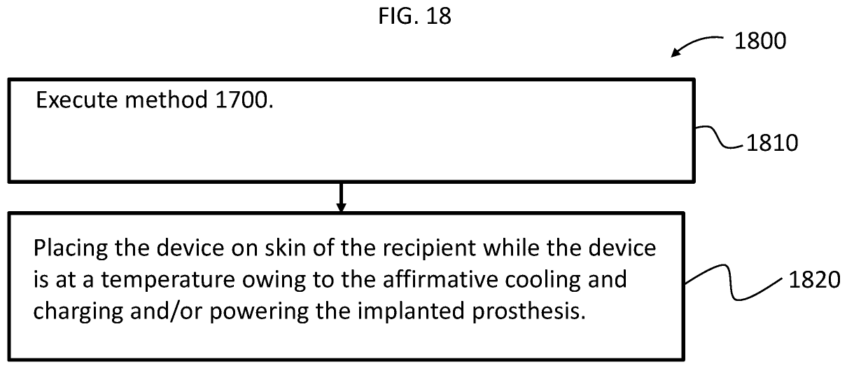

FIG. 18 presents another exemplary method, method 1800, which includes executing method 1700. The method also includes method action 1820, which includes placing the device on skin of the recipient while the device is at a temperature owing to the affirmative cooling and charging and/or powering the implanted prosthesis. In an exemplary embodiment, the action of placing the device on skin is executed within a time period less than or equal to 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 4, 4.5, 5, 5.5 or 6 minutes or any value or range of values there between in 0.01 minute increments, from the end of charging of the device and/or from a point in time where an internal temperature of the device has stopped increasing (and immediately prior to the halting of the temperature increase, the temperature was increasing). In an exemplary embodiment, these time periods would otherwise be time periods where the device would be hot, including too hot, for comfort and/or safety vis-à-vis contact with the skin of the person, but for the affirmative cooling, the device is not as hot as it otherwise would be. Thus, in an exemplary embodiment, the device has a temperature that falls within the various standards for medical devices even after a charging regime that would otherwise result in the device being at a temperature that would violate the standards.

In an exemplary embodiment, the skin interface surface is, after one or more of the aforementioned recharging scenarios, at a temperature at least, or equal to 0.5, 1, 1.5, 2, 2.5, 3, 3.5, 4, 4.5, 5, 5.5, 6, 6.5, 7, 8, 9, 10, 11, 12, 13, 14, 15, 16, 17, 18, 19, 20, 21, 22, 23, 24, 25, 26, 27, 28, 29 or 30 or any value or range of values therebetween in 0.1° C. increments, below that which would otherwise be the case in the absence of the application of the cooling/heat transfer teachings detailed herein all other things being equal.

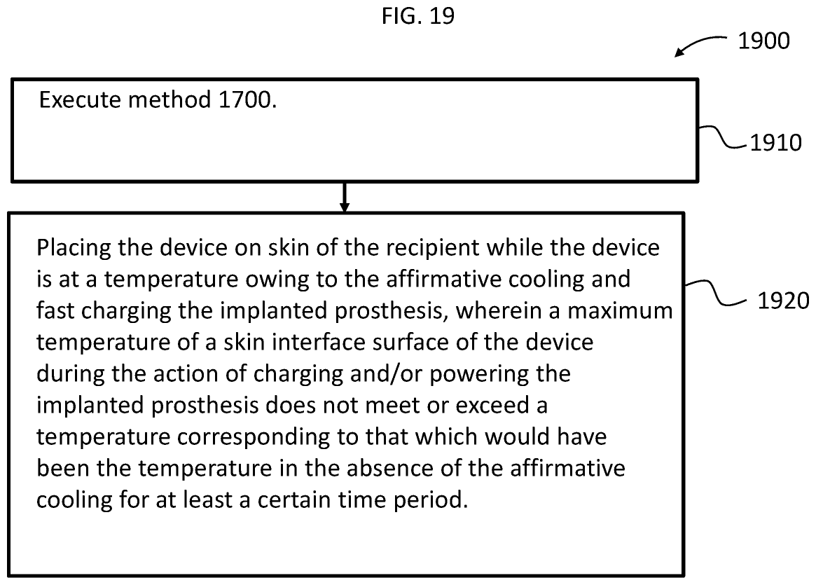

FIG. 19 is a flowchart for a method, method 1900. Method 1900 includes method action 1910, which includes executing method 1700. Method 1900 further includes method action 1920, which includes placing the device on skin of the recipient while the device is at a temperature owing to the affirmative cooling and charging, including in some embodiments, fast charging, the implanted prosthesis. In this method, a maximum temperature of a skin interface surface of the device during the action of charging and/or powering the implanted prosthesis does not meet or exceed a temperature corresponding to that which would have been the temperature in the absence of the affirmative cooling for at least a certain time period after the commencement of the charging, where the certain time period is at least or equal to 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or 60 or more minutes or any value or range of values therebetween in 0.1 minute increments. In an exemplary embodiment, the skin interface surface is, after one or more of the aforementioned implant recharging scenarios, at a temperature at least, or equal to 0.5, 1, 1.5, 2, 2.5, 3, 3.5, 4, 4.5, 5, 5.5, 6, 6.5, 7, 8, 9, 10, 11, 12, 13, 14, 15, 16, 17, 18, 19, 20, 21, 22, 23, 24, 25, 26, 27, 28, 29 or 30 or any value or range of values therebetween in 0.1° C. increments, below that which would otherwise be the case in the absence of the application of the cooling/heat transfer teachings detailed herein all other things being equal.

In an exemplary embodiment, the cooling of the charging device results in the skin interface surface being at a temperature of no more than or at least equal to 25, 26, 27, 28, 29, 30, 31, 32, 33, 34 or 35 degrees Celsius or any value or range of values therebetween in 0.1° C. where the ambient temperature at the location where the recharging takes place is at least 3, 4, 5, 6, 7, 8, 9, 10, 11, 12, 13, 14 or 15 or more degrees Celsius higher than that temperature.

Is briefly noted that while the teachings detailed herein or otherwise transferring heat away from the external component while the external component is recharged, other embodiments can include cooling a component that is already charged. By way of example only and not by way of limitation, the charging device can be charged and be ready to be used, and could have been this way for as many as two or three or four hours or even a day or two or longer prior to the time that the charging devices needed or otherwise utilized to charge the implant. However, owing to the ambient conditions or otherwise owing to the desire to have a very fast charging of the implant device, there can be utilitarian value with respect to cooling the charging device prior to using the charging device to charge the implant. The goal is to avoid overheating the skin at the location where charging takes place. Accordingly, anything that avoid such can have utilitarian value.

In an exemplary embodiment, the heat transfer/cooling teachings detailed herein can result in the ability of the charging device to be utilized to charge an implant at a rate of charge that is at least 1.5, 2, 2.5, 3, 3.5, 4, 4.5, 5, 5.5 or 6 times or more than that which would otherwise have been the case, all other things being equal, while keeping the skin temperature at the location where the charging device contacts the skin below a skin temperature of 43, 42, 41, 40, 39, 38, 37, 36, or 35 degrees Celsius, with the charging takes place for at least 10, 15, 20, 25, 30, 35, 40, 45, 50, 55, or 60 minutes.

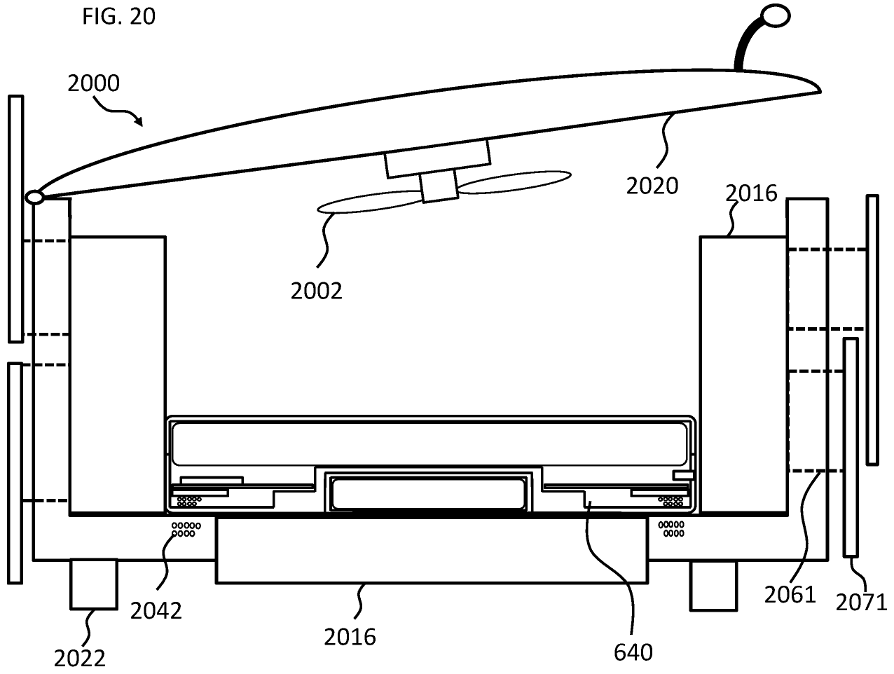

FIG. 20 provides an exemplary device 2000 that can enable some of the teachings detailed herein during the action of charging an external component that is utilized to charge or otherwise provide power to an implantable component, such as by way of example only and not by way of limitation, the external component 640, which is shown in FIG. 20 as being located in the charging device 2000 in a position for charging. The charging device 2000 is configured to inductively charge the external component utilizing an inductance coil charging device 2042, which charging device can correspond to or otherwise have the components that are similar to or otherwise analogous to the components that are utilized by the external component to charge the implant. That said, in an alternate embodiment, a hardwired system including a plug that plugs into the external component 640 can be utilized to charge the external component 640. That is, inductance charging is but one option, and a more traditional method of charging utilizing the direct flow of electrons the battery of the external component can be utilized in some other embodiments. Moreover, it is noted that the coils used for inductance communication with the implant may not necessarily be used for recharging. In some embodiments, a second coil for recharging is present and/or additional circuitry is used to convert the AC current to the DC current (so that a different recharging frequency (from the power transmission to the implant frequency) can be used, for example). The additional coil can be co-located with the transfer coil, or could be located away from the coil (e.g., on the opposite side of the headpiece—in which case external component 640 would be shown in FIG. 2 upside down from that shown).

In an exemplary embodiment, the device is configured to recharge a power storage portion of the prosthesis component. The power storage portion can be battery cells that are configured to be recharged. In an exemplary embodiment, the prosthesis component is a battery, such as battery 252 of the BTE device 1040 of FIG. 4. In an exemplary embodiment, the prosthesis component is an external component of a hearing prosthesis in its entirety (e.g., component 640 as seen in FIG. 20), and in another exemplary embodiment, it is the prosthesis charging device (in its entirety), such as the device used to recharge a totally implantable hearing prosthesis, such as for example the variation of the embodiment of FIG. 6 (where there is no sound capture element 526 and no sound processor—the device is purely a device configured to recharge the implanted portion) or FIG. 11 (by way of example only). The device is configured to cool an assembly of which the power storage portion is apart (e.g., the assembly of battery 252, the assembly of the off the ear charger, etc.).

Some embodiments of the charging device are configured to charge one or more of the aforementioned external components and/or batteries detailed herein in accordance with one or more of the various recharging regimes detailed herein. The charging device 2000 can be configured to plug into a standard alternating current outlet so as to obtain power for the operation of the charging device. The charging device 2000 is configured with a lid 2020 that enables the inside of the charging apparatus to be isolated from the ambient environment. This can have utilitarian value with respect to this particular embodiment in which the charging device 2000 is unique in that the charging device also enables cooling of the external component 640 during charging and/or while the external component is located therein. In this regard, the exemplary charging device 2000 seen in FIG. 20 includes three thermoelectric coolers 2016. As seen, two of these have heatsinks 2061 that lead to radiator devices 2071 so as to transfer heat from inside the enclosure to outside the enclosure, thus cooling the external component 640. The thermoelectric cooler 2016 at the bottom is seen extending all the way through the enclosure wall and does not have a heatsink per se. As seen, support pedestals 2022 are located on the bottom of the charging device 2000 so that air can flow under the bottom of the thermoelectric cooler 2016. To be clear, in the embodiment shown in FIG. 20, the “cool side” of the thermoelectric coolers are located facing the external component 640, and the“hot side” of the thermoelectric coolers are located facing the outside of the enclosure, away from the external component. This enables heat to be transferred from the external component or otherwise from the inside of the enclosure to the outside of the enclosure, thus effectively refrigerating the inside of the charging device.

Also as seen, the charging device of FIG. 20 includes a fan 2002 which can be utilized to transfer heat from the external component 640. In an exemplary embodiment, the charging apparatus can be configured with inlet and outlet ports so that airflow through the enclosure can be enhanced or otherwise enabled. That said, in an alternate embodiment, such as where the enclosure is in a semi-sealed configuration (akin to how a refrigerator enclosure is sealed), the fan 2002 can be utilized to move air within the enclosure so that there is airflow across the “cold sides” of the thermoelectric cooling devices.

It is noted that while the charging device of FIG. 20 relies on thermoelectric cooling and/or convection heat transfer, in an alternative embodiment, a refrigeration system utilizing compressed and expanded gas (the Carrier refrigeration cycle) can be utilized instead or in addition to the embodiments seen in FIG. 20. Note also that in some embodiments, a more technologically simple arrangement might be utilized external component. In an exemplary embodiment, pre-cooled substances, such as an ice pack, can be placed in the enclosure to cool the enclosure and otherwise extract heat from the external component 640. In an exemplary embodiment, this icepack can be a preformed component (e.g., it is not a bag of ice, but instead a plastic container containing a substance that is easily cooled in a repeatable manner) that can be put in a freezer or otherwise maintained in a freezer and then taken out and utilized when recharging is to be implemented. In an exemplary embodiment, the icepack can be placed on top of the lid, and the lid can have a preformed structure that can receive the icepack, and air vents through the lid can suck air from outside the enclosure into the enclosure, which air will be cooled while the air passes over/around the icepack, thus drawling cold air into the enclosure, and thus cooling the external component 640 during charging.

Thus, in an exemplary embodiment, there is a method that includes executing method 1700 with the additional action of obtaining access to a charging apparatus configured to interface with the device to be recharged (the external component) and recharge the power storage component, wherein the action of recharging the power storage component is executed using the charging apparatus that is configured to affirmatively cool the device during the charging and/or before the charging, and the charging apparatus is used to affirmatively cool the device. Here, the charging apparatus to which access is obtained can be the apparatus of FIG. 20. Thus, in some embodiments, the charging apparatus to which access is obtained includes a container. In this regard, the methods can further include the method actions of placing the device to be recharged into the container so that the device is completely enclosed in the container, and lowering the temperature of the device while the device is in the container.

In some embodiments, the interior of the container is cooled to a temperature of 33, 32, 31, 30, 29, 28, 28, 27, 26, 25, 24, 23, 22, 21, 20, 19, 18, 17, 16, 15, 14, 13, 12, 11, or 10 degrees Celsius or lower, and such is the case for at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, or 180 minutes or longer, all while the device being charged is in the container being charged.

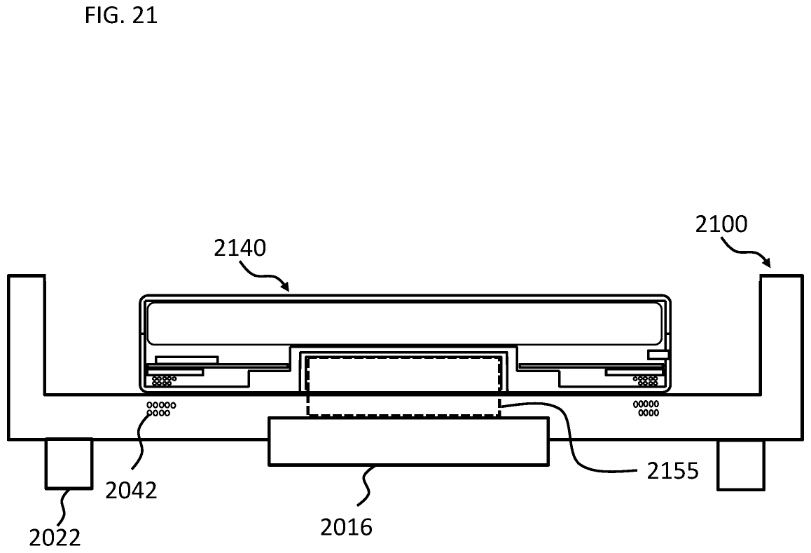

FIG. 21 presents another exemplary embodiment of a charger 2100, for an external component, here, external component 2140. In this embodiment, charger 2100 includes a heatsink 2155 that extends from the thermoelectric cooler 2016 as shown. The heatsink 2155 is sized and dimensioned to fit into the external component 2140 as shown. In this regard, the external component includes a coupling to removably attached to the external component to a separate heat transfer device. This coupling interfaces with the heatsink 2155. He can be transferred from the external component 2140, including from inside the external component, directly to the heatsink 2155. This can enhance heat transfer during recharging. This heat transfer can be executed during recharging. In an exemplary embodiment, the magnet can be removed from the external component 2140 to provide access to the coupling.

It is noted that the external components without the heat transfer systems/cooling systems do not include part of the specification, and should be considered prior art. Thus, embodiments include means for inductance power transfer communication, which include for example an inductance coil established by heat pipes, and because the traditional/prior art inductance power transfer communications do not form part of the innovative features, but are modified with innovative features herein/are used with the innovative features, means for inductance power transfer communication does not include these prior art devices. Such is also the case with respect to heat transfer deices/apparatuses/systems—the mere fact that any device may transfer heat does not corresponds to a heat transfer device, etc.

In an exemplary embodiment, the devices that are dedicated prosthesis charging device are configured such that when they are inductively coupled to a prosthesis charging component for inductive charging of that component, the devices can fully recharge the component from at least a 50, 55, 60, 65, 70, 75, 80, 85, 90, 95 or 100% depleted battery state in a time period that is at least 20, 25, 30, 35, 40, 45, 50, 55, 60, 65 or 70% or any value or range of values therebetween in 1% increments shorter than that which would otherwise be the case in the absence of the availability of the functionality of heat transfer arrangements detailed herein such as those of the charging device, all other things being equal, such that the skin interface component is not above 37, 38, 39, 40, 41, 42, or 43 degrees Celsius at the end of the time period when an ambient temperature of the device is at least 35, 36, 37, 38, 39, 40, 41 or 42 degrees Celsius or any value or range of values therebetween in at least 0.1° C. increments in a shaded still air condition.

In an exemplary embodiment, the charging devices include circuitry, such as microprocessors, configured to implement fast charging versus standard charging (they are configured to implement both—the devices are configured to utilize the circuitry so as to implement such charging regimes).

It is noted that any method detailed herein also corresponds to a disclosure of a device and/or system configured to execute one or more or all of the method actions associated with the device and/or system as detailed herein. In an exemplary embodiment, this device and/or system is configured to execute one or more or all of the method actions in an automated fashion. That said, in an alternate embodiment, the device and/or system is configured to execute one or more or all of the method actions after being prompted by a human being. It is further noted that any disclosure of a device and/or system detailed herein corresponds to a method of making and/or using that device and/or system, including a method of using that device according to the functionality detailed herein.

It is further noted that any disclosure of a device and/or system detailed herein also corresponds to a disclosure of otherwise providing that device and/or system.

It is also noted that any disclosure herein of any process of manufacturing and/or providing a device corresponds to a device and/or system that results therefrom. It is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A device, comprising:
an inductive power transmission apparatus, wherein
the device includes a dedicated heat transfer arrangement configured to transfer away from the device heat that is generated when transferring power using the device, and
the device is configured to transmit inductance power transcutaneous into a person.

2. The device of claim 1, wherein:
the inductive power transmission apparatus includes a heat pipe that is part of a resonant tank of a closely coupled inductive link in a wireless power transmission system.

3. The device of claim 1, wherein:
the inductive power transmission apparatus includes an induction coil that is a heat pipe.

4. The device of claim 1, wherein:
the device is at least part of an external component of a prosthesis system that utilizes transcutaneous inductive power transfer to power an implanted component.

5. The device of claim 1, wherein:
the device is configured to induce movement of air through the device beyond that which would occur as a result of normal convention to enhance heat transfer from the dedicated heat transfer arrangement.

6. The device of claim 1, wherein:
the inductive power transmission apparatus is also an inductive communication apparatus.

7. The device of claim 1, wherein:
the heat transfer arrangement includes a coupling to removable attach the device to a separate heat transfer device.

8-21. (canceled)

22. A device, comprising:
an inductive power transmission sub-system configured to transfer power to an implanted medical device;
a skin interface surface; and
a cooling sub-system configured to cool the skin interface surface.

23. The device of claim 22, wherein:
the system for cooling the skin interface surface is a means for cooling the skin interface surface.

24. The device of claim 22, wherein:
the device is configured so that power can be transferred at a rate of at least twice that which would be the case in the absence of the system for cooling the skin interface surface while maintaining the same temperature thereof, all other things being equal.

25. The device of claim 22, wherein:
the cooling sub-system is integrated with the inductive power transmission sub-system.

26. The device of claim 22, wherein:
the device is configured to transfer heat with a part that also transmits power, thereby cooling the skin interface surface.

27. The device of claim 22, wherein:
the device is an off the ear charging device or an off the ear sound processor; and
the cooling sub-system is configured to transfer heat from the skin interface surface to a location on the device opposite the skin interface surface.

28. (canceled)

29. The device of claim 22, wherein:
the inductive power transmission sub-system includes an inductance coil that is a heat pipe.

30. A device, comprising:
a battery charging apparatus; and
a cooling device, wherein
the device is a dedicated prosthesis component charging device configured to recharge a power storage portion of the prosthesis component while cooling an assembly of which the power storage portion is apart.

31. The device of claim 30, wherein:
the device is a dedicated hearing prosthesis component charging device.

32. The device of claim 30, wherein:
the device includes a means for cooling the prosthesis component.

33. The device of claim 30, wherein:
the device is a fast charger for an implant charger for a totally implantable cochlear implant.

34. The device of claim 30, wherein:
the device includes a compartment sized and dimensioned to receive the prosthesis component; and
the device is configured to reduce an air temperature within the compartment by at least 5 degrees Celsius relative to an ambient air temperature of air in which the device is located.

35. The device of claim 30, wherein:
the prosthesis component is inductively coupled to the device; and
the device is configured to fully recharge the prosthesis component from at least a 90% depleted battery state in a time period that is at least 30% shorter than that which would otherwise be the case in the absence of the availability of the functionality of the cooling device, all other things being equal, such that the skin interface component is not above 41 degrees Celsius at the end of the time period when an ambient air temperature of the device is at least 35 degrees Celsius in a shaded still air condition.

36-38. (canceled)
